SEQUENCE LISTING (15 sequences)

SEQ ID NO: 1 - DNA, 43 bp, Artificial sequence
  Amplification primer
accgatgacgacgacaaggccatggctatgactgatctgaaag

SEQ ID NO: 2 - DNA, 38 bp, Artificial sequence
  Amplification primer
tggttgaggagaagccaagcttagtagctgctggcgct

SEQ ID NO: 3 - DNA, 43 bp, Artificial sequence; identical to SEQ ID NO: 1
  Amplification primer

SEQ ID NO: 4 - DNA, 35 bp, Artificial sequence
  Amplification primer
tggttgaggagaagccaagcttagctgctggcgct

SEQ ID NO: 5 - DNA, 21 bp, Artificial sequence
  Cloning primer
gacgacgacaagatgcatatg

SEQ ID NO: 6 - DNA, 21 bp, Artificial sequence
  Cloning primer
gaggagaagcccggtttagta

SEQ ID NO: 7 - DNA, 27 bp, Artificial sequence
  Mutagenesis primer
gcgggcggcgtggaaactgcggaagat

SEQ ID NO: 8 - DNA, 27 bp, Artificial sequence
  Mutagenesis primer
actttccgcagtttccacgccgcccgc

SEQ ID NO: 9 - DNA, 27 bp, Artificial sequence
  Mutagenesis primer
tttggcgcttccgaactgctcgcaagc

SEQ ID NO: 10 - DNA, 27 bp, Artificial sequence
  Mutagenesis primer
gcttgccagcagttcggaagcgccaaa

SEQ ID NO: 11 - DNA, 27 bp, Artificial sequence
  Mutagenesis primer
cgctttggcgctgacagcctgctggca

SEQ ID NO: 12 - DNA, 21 bp, Artificial sequence
  Mutagenesis primer
tctaccggtgaagtggctgtg

SEQ ID NO: 13 - DNA, 21 bp, Artificial sequence
  Mutagenesis primer
cacagccacttcaccggtaga

SEQ ID NO: 14 - DNA, 27 bp, Artificial sequence
  Mutagenesis primer
aaaccggcgggcgaagtgcgtactgcg

SEQ ID NO: 15 - DNA, 27 bp, Artificial sequence
  Mutagenesis primer
cgcagtacgcacttcgcccgccggttt

(19) United States
(12) Patent Application Publication
(10) Pub. No.: US 2007/0015260 A1
(43) Pub. Date: Jan. 18, 2007

(54) Synthesis of synthons for the manufacture of bioactive compounds

(75) Inventors: Chi-Huey Wong, Rancho Santa Fe, CA (US); Junjie Liu, San Diego, CA (US); Grace DeSantis, San Diego, CA (US); Mark Burk, San Diego, CA (US)

Correspondence Address: DLA PIPER US LLP, 4365 EXECUTIVE DRIVE, SUITE 1100, SAN DIEGO, CA, 92121-2133, US

(73) Assignees: DIVERSA CORPORATION; THE SCRIPPS RESEARCH INSTITUTE

(21) Appl. No.: 11/481,653

(22) Filed: Jul. 5, 2006

Related U.S. Application Data
(62) Division of application No. 10/390,544, filed on Mar. 14, 2003, now abandoned.
(60) Provisional application No. 60/364,641, filed on Mar. 14, 2002.

(51) Int. Cl. C08B 37/00; C12P 19/02; C12N 9/24

(52) U.S. Cl. 435/105; 536/124; 435/200

(57) ABSTRACT

The present invention is based on the discovery that 2-deoxyribose-5-phosphate aldolase (DERA, EC 4.1.2.4) and variants thereof can be used to catalyze sequential asymmetric aldol reactions between a wide variety of donor and acceptor aldehydes. The reaction products typically contain at least two new stereogenic centers and can be produced in enantiomerically pure form. As such, DERA catalyzed asymmetric aldol chemistry can be exploited to produce synthons for the synthesis of a variety of bioactive molecules.

This application claims the benefit of priority under 35 U.S.C. §119(e) of U.S. Ser. No. 60/364,641, filed Mar. 14, 2002, and is a divisional application of, and claims the benefit of priority to, U.S. Ser. No. 10/390,544, filed Mar. 14, 2003, under 35 U.S.C. §§120 and 121. The entire contents of applications U.S. Ser. Nos. 60/364,641 and 10/390,544 is incorporated herein by reference.

This invention was made in part with government support under Grant No. GM44154 awarded by the National Institutes of Health. The United States government may have certain rights in this invention.

### BACKGROUND OF THE INVENTION

1. Field of the Invention

The invention relates generally to the use of enzymes in organic synthesis, and more particularly to aldolase-catalyzed asymmetric synthesis for the production of bioactive compounds.

2. Background Information

Enzymes are now widely exploited as catalysts in asymmetric organic synthesis, due to their exquisite chemo-, regio- and stereo-specificity. The aldolases are a particularly useful class of enzymes because these enzymes catalyze C—C bond formation with high stereoselective control at the newly formed stereogenic centers. More than 20 aldolase structures have been reported to date and most contain a common α8β8 barrel structural motif. Recent advances inmolecular genetics, protein engineering, and site-specific modification of enzymes have further expanded the scope of enzyme catalysis with regard to synthetic applications.

The enzyme 2-deoxyribose-5-phosphate aldolase (DERA, EC 4.1.2.4), a Schiff base forming type I class aldolase, catalyzes the reversible aldol reaction of acetaldehyde and D-glyceraldehyde 3-phosphate (G3P) to form D-2-deoxyribose-5-phosphate (DRP). The enzyme has been overexpressed in Escherichia coli, and its structure and catalytic mechanism have been determined at the atomic level. However, the potential utility of this particular aldolase in asymmetric organic synthesis has not yet been fully realized.

In addition, expanding the range of unnatural substrates that aldolases will accommodate as well as overcoming their instability and high cost is crucial to further increasing the scope of their synthetic application. The invention addresses these issues and further provides related advantages.

### SUMMARY OF THE INVENTION

The present invention is based on the discovery that 2-deoxyribose-5-phosphate aldolase (DERA, EC 4.1.2.4) and variants thereof can be used to catalyze sequential asymmetric aldol reactions between a wide variety of donor and acceptor aldehydes. The reaction products typically contain at least two new stereogenic centers and can be produced in enantiomerically pure form. As such, DERA catalyzed asymmetric aldol chemistry can be exploited to produce synthons for the synthesis of a variety of bioactive molecules.

In one aspect of the invention, there are provided methods for producing enantiomerically pure pyranoses. Such methods can be performed, for example, by contacting a first achiral aldehyde, a second achiral aldehyde, and a third achiral aldehyde with 2-deoxyribose-5-phosphate aldolase (DERA) or a variant thereof under conditions suitable to facilitate sequential asymmetric aldol reactions, wherein a first aldol reaction between the first and second achiral aldehydes forms a first reaction product, wherein a second aldol reaction between the first reaction product and the third achiral aldehyde forms a second reaction product, wherein the second reaction product spontaneously undergoes an intramolecular cyclization reaction to form an enantiomerically pure pyranose.

In another aspect of the invention, there are provided methods for producing epothilone precursor molecules. Such methods can be performed, for example, by contacting an acceptor β-hydroxy-aldehyde with at least one donor aldehyde in the presence of 2-deoxyribose-5-phosphate aldolase (DERA) or a variant thereof under conditions suitable to facilitate sequential asymmetric aldol reactions, thereby producing epothilone precursor molecules.

In another aspect, there are provided methods for producing atorvastatin precursor molecules. Such methods can be performed, for example, by contacting a β-hydroxy-aldehyde with an azide-containing acceptor aldehyde in the presence of a DERA variant, under conditions suitable to facilitate sequential asymmetric aldol reactions, thereby producing atorvastatin precursor molecules.

In another aspect, there are provided isolated 2-deoxyribose-5-phosphate aldolases having any one of the following mutations: K172E, G205E, R207E, S238D, or S239E, and polynucleotides encoding the invention aldolases.

In still another aspect, there is provided an isolated E. coli having the characteristics of Δace, adhC, DE3.

In a further aspect of the invention, there are provided methods for identifying 2-deoxyribose-5-phosphate aldolase (DERA) variants having expanded substrate specificity as compared to wild-type DERA polypeptides. Such methods can be performed, for example, by culturing a prokaryote transformed with a polynucleotide encoding a DERA variant, wherein the prokaryote either utilizes acetaldehyde as a sole-carbon source or requires acetaldehyde supplementation for growth, whereby growth of the prokaryote is indicative of the presence of a 2-deoxyribose-5-phosphate aldolase (DERA) variant having expanded substrate specificity as compared to wild-type DERA polypeptide.

### BRIEF DESCRIPTION OF THE DRAWINGS

FIG. 1 illustrates the mechanism of DERA catalyzed aldol reaction between the natural donor acetaldehyde and acceptor D-glyceraldehyde-3-phosphate to generate D-2-deoxyribose-5-phosphate.

FIG. 2 illustrates an overlap of eight known aldolase αβ barrels showing the Lys residue for Schiff base formation.

FIG. 3 illustrates a stereoview of the active site of the DERA carbinolamine complex.

FIGS. 4 A-D illustrate DERA product modeling based on the Schiff base complex structure (PDB code 1JCJ).

FIG. 5 illustrates DERA catalyzed synthesis of designed substrates.

FIG. 6 illustrates that metabolically engineered SELECT (Δace, adhC, DE3) E. coli strain requires 2-carbon supplementation for cell growth.

FIG. 7 is a schematic illustrating proof of concept for the selection protocol using SELECT.

### DETAILED DESCRIPTION OF THE INVENTION

In one aspect, the invention provides methods for producing enantiomerically pure pyranoses. Such a method can be performed, for example, by contacting a first achiral aldehyde, a second achiral aldehyde, and a third achiral aldehyde with 2-deoxyribose-5-phosphate aldolase (DERA) or a variant thereof under conditions suitable to facilitate sequential asymmetric aldol reactions, wherein a first aldol reaction between the first and second achiral aldehydes forms a first reaction product, wherein a second aldol reaction between the first reaction product and the third achiral aldehyde forms a second reaction product, wherein the second reaction product spontaneously undergoes an intramolecular cyclization reaction to form an enantiomerically pure pyranose. This sequential aldol/cyclization chemistry is outlined in Scheme 1.

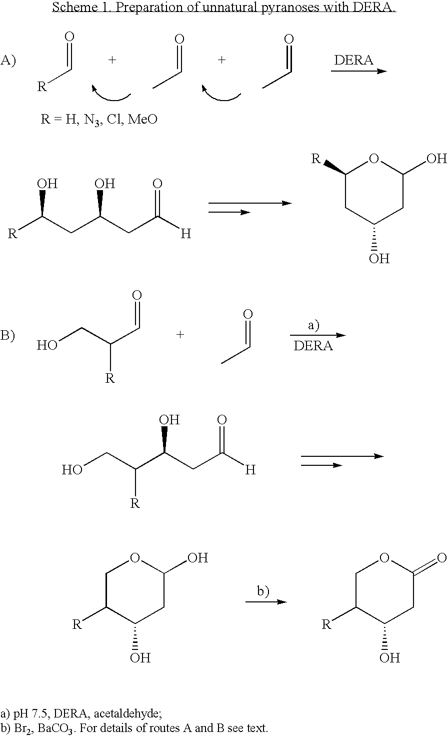

In this sequential reaction, the first aldol product acts as a substrate for the second aldol reaction to give an enantiomerically pure 3,5-dihydroxyaldehyde which then cyclizes to form a stable pyranose, thus driving the reaction toward condensation. Since these 1,3-polyol systems are useful synthons, the scope of this enzymatic methodology was examined further. One strategy is to exploit β-hydroxy-aldehydes as acceptors (Scheme 1) to generate products which cyclize to form stable hemiacetals, thus driving the reaction toward condensation. The hemiacetal can be further oxidized to give a lactone. Indeed, the oxidation sometimes makes the purification much easier, and more importantly, the lactone can be further transformed to other useful synthons. Several different substrates have been tested and the results are summarized in Table 1.

TABLE 1
Aldol condensation catalyzed by DERA.

| Entry | Acceptor | Donor | t [days] | Product | Yield [%] |
|---|---|---|---|---|---|
| 1 | 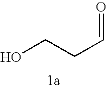 | 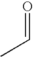 | 3 | 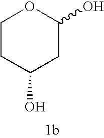 | 65 |
| 2 | 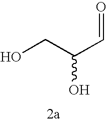 | 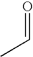 | 3 | 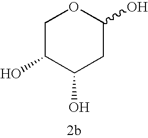 | 60^{[a]} |
| 3 | 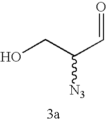 | 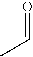 | 3 | 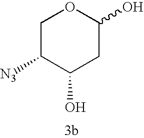 | 47^{[a]} |
| 4 | 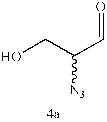 | 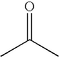 | 6 | 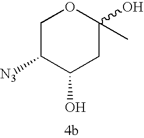 | 28^{[a]} |
| 5 | 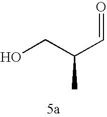 | 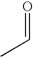 | 3 | 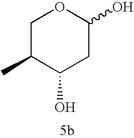 | 48^{[b]} |
| 6 | 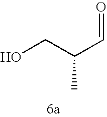 | 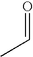 | 6 | 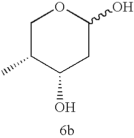 | trace^{[b]} |
| 7 | 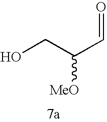 | 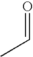 | 6 | 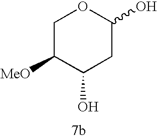 | 22^{[a]} |

^{[a]}Based on the reactive enantiomers.
^{[b]}Total yield for two steps from protected aldehyde.

The configuration of C2 in the acceptor aldehydes effects the outcome of the enzymatic reaction.

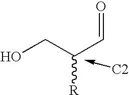

It was found that D isomers were overwhelmingly preferred over L isomers when polar groups (e.g., R═OH, N₃) were at this position; when racemic acceptor aldehydes were used, only the D isomer products were formed (Table 1, entries 24). On the contrary, an opposite enantioselectivity is observed when a hydrophobic group is at the C2 position (Table 1, entries 5-7): 5a afforded lactone 5b in 48% yield after two steps, while its enantiomer 6a only gave 6b in trace amounts, and racemic aldehyde 7a only produced 7b. Molecular modeling based on the structure of DERA reveals a hydrophilic binding pocket composed of Thr170 and Lys172 for the OH group at C2 and a hydrophobic pocket for the H atom at C2. A switch of the binding was observed for 5a and 7a in which the methyl and the methoxy groups are in the hydrophobic pocket, which results in a change of enantioselectivity.

The 1,3-polyol systems prepared from the enzymatic reaction serve as useful synthons. One example involves the stereoselective C2 alkylation of β-hydroxylactone with an alkyl bromide under chelation control directed by the β-hydroxy group (Scheme 2).

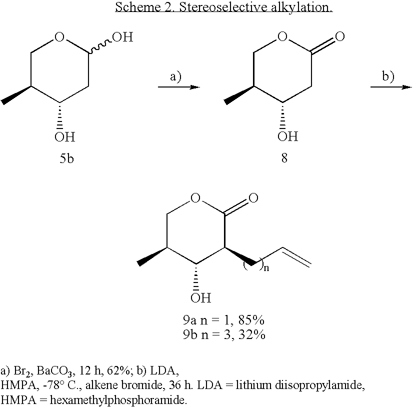

This reaction can provide more diversified pyranoses after reduction of the lactones and generate additional useful intermediates for organic synthesis. In the alkylation experiment, the other diastereomers were not detected. The relative configuration of 9a was unequivocally confirmed by NMR experiments.

The availability of both intermediates 9a and 9b permitted us to choose either the Suzuki coupling or olefin metathesis strategy to prepare epothilones as potential anticancer agents. Since allyl bromide is more active and gives 9a in a higher yield, the Suzuki coupling strategy was chosen for the construction of the C12-C13 Z double bond (Scheme 3). In addition to 9a, compound 11 prepared by DERA was also used as a key synthon.

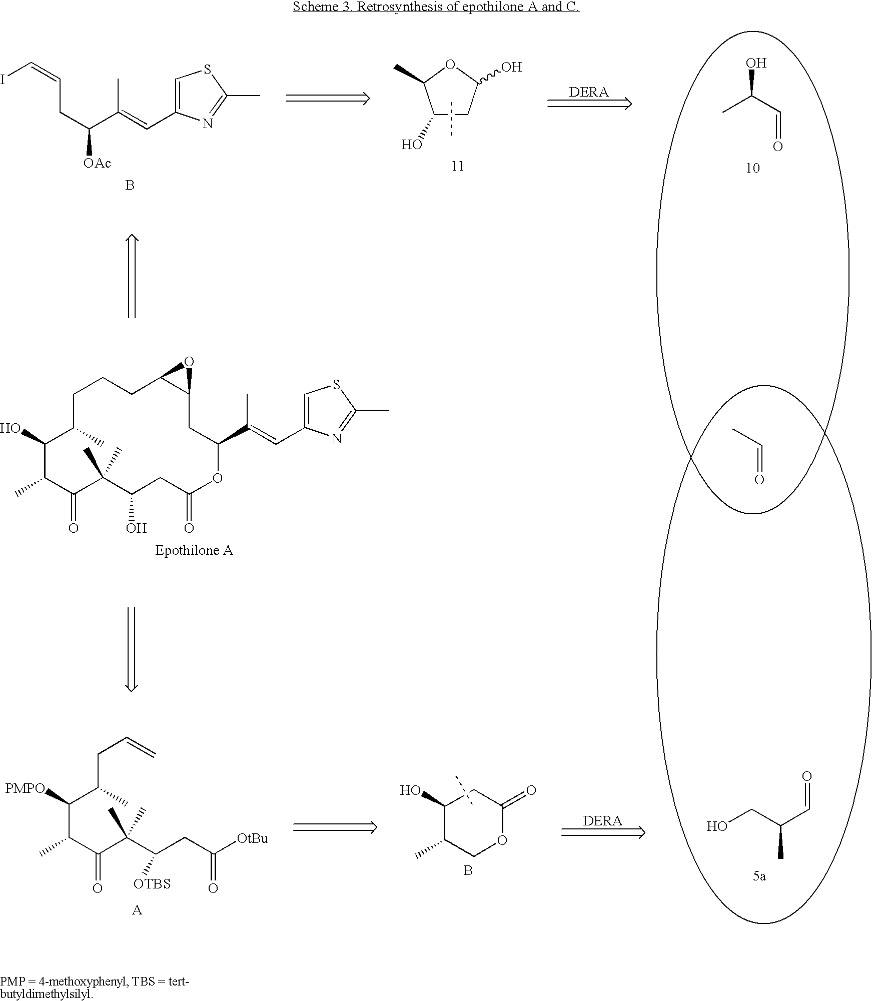

In our synthesis of fragment A (Scheme 4), the lactone ring of 9a was first opened to afford diol 12, which was then protected as the PMP acetal. After reduction by LiAlH₄, the hydroxy was removed by mesylation followed by reduction, both in excellent yield. Regioselective cleavage of the PMP protecting group in 13 with DIBAL in toluene gave the primary alcohol as the only product, which was oxidized with Dess-Martin periodinane to give aldehyde 14. Compound 14 was then condensed with tert-butyl isobutyrylacetate to give compound 15 in 70% yield (d.r. 8:1). Stereoselective reduction with Me₄NBH(AcO)₃ resulted in the formation of the desired diol (d.r. 10:1). Regioselective silylation of the β-hydroxy group followed by oxidation gave fragment A.

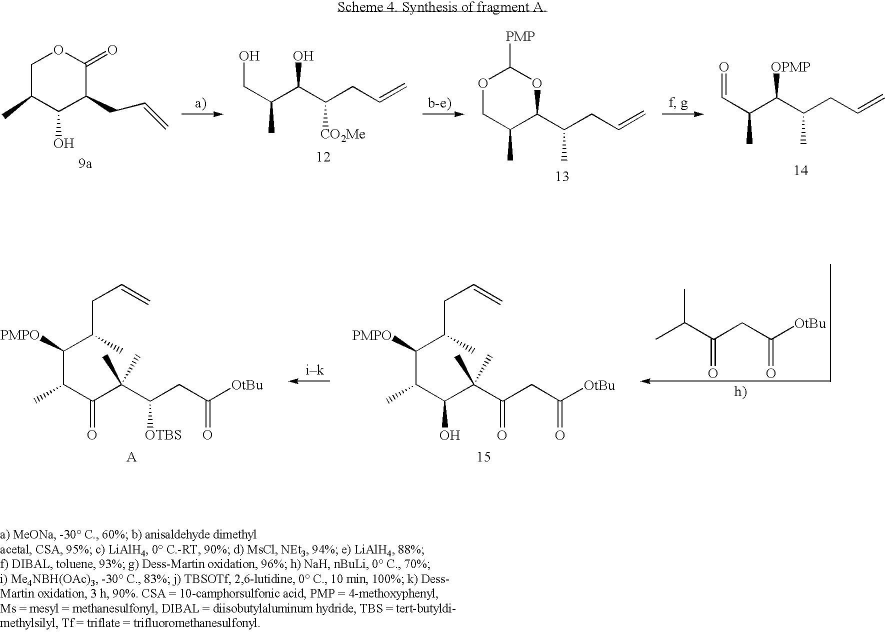

Because the configuration of C2 in 16 is not essential in our synthetic route (Scheme 5), racemic lactaldehyde acetal 16 was used in our current synthesis. Interestingly, we found that only the D isomer was accepted as a substrate for DERA and no L isomer product was detected in our experiment. The preparation of fragment B is rather straightforward (Scheme 5).

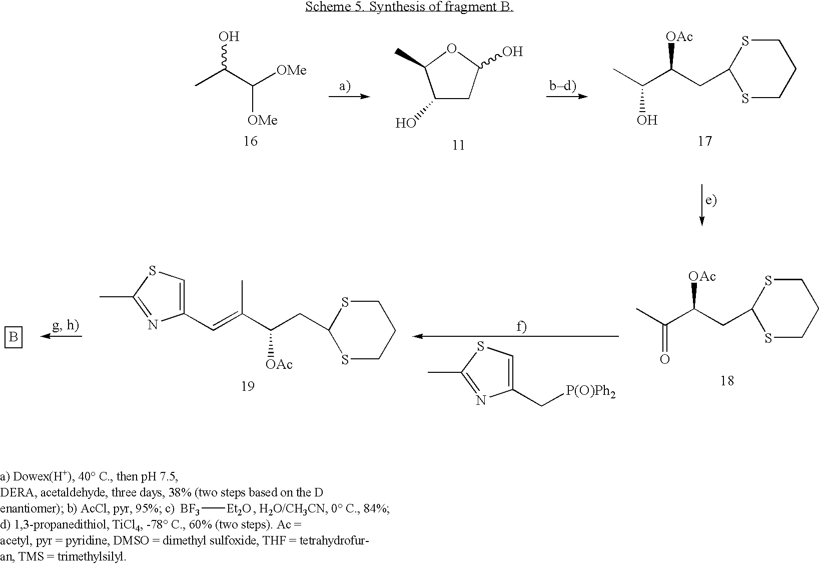

The β-hydroxy group of was selectively protected and the hemiacetal was treated with 1,3-propanedithiol to afford the dithiane 17, which was oxidized to ketone 18 in 95% yield. Wittig reaction of 18 with a phosphine oxide afforded 19. Following deprotection of the dithiane with Hg(OCl₄)₂, the aldehyde product was directly coupled with (Ph3P⁺CH₂I)I⁻ to afford fragment B in 60% yield for the two steps.

The Suzuki coupling of fragments A and B proceeded smoothly as described by Danishefsky, et. al., to afford 20 (Scheme 6).

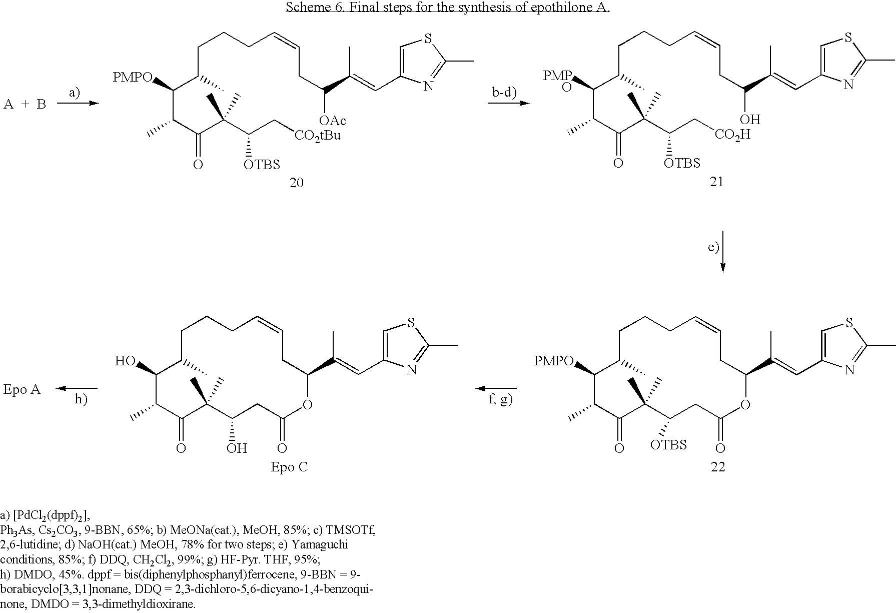

After the acetyl and tert-butyl ester protecting groups were removed, the hydroxy acid 21 was subject to Yamaguchi macrolactonization conditions to afford the intermediate 22. The PMP and TBS protecting groups were removed with DDQ and HF pyr, respectively, to furnish epothilone C. Epoxidation with a freshly prepared solution of 1,3-dimethydioxirane (DMDO) afforded synthetic epothilone A with physical properties ([α]_{D}, ¹H, ¹³C NMR, MS, IR) identical to the reported data.

In summary, a new strategy for the synthesis of unnatural pyranose synthons has been developed, through enzymatic reactions catalyzed by DERA. This strategy is very convergent and effective. Coupled with P-hydroxy-directed highly stereoselective alkylation, diversified 1,3-poyols can be prepared. Their application to natural product synthesis has been illustrated by the concise total synthesis of epothilones A and C.

In a further aspect of the invention, there are provided methods for identifying 2-deoxyribose-5-phosphate aldolase (DERA) variants having expanded substrate specificity. Indeed, it is desirable to expand the specificity of DERA beyond its natural substrate D-2-deoxyribose-5-phosphate (DRP) and improve its activity with nonphosphorylated substrates.

Numerous methods to alter enzyme properties now exist. These include, for example, solvent or substrate engineering, enzyme adsorption and covalent chemical modifications of enzymes. More recently, site-directed mutagenesis and random mutagenesis approaches to alter enzyme specificity have been exploited. The former often requires a detailed understanding of the enzyme's catalytic mechanism, substrate specificity determinants and tertiary structure. By contrast, random mutagenesis approaches do not require prior understanding of specificity determinants nor knowledge of the structure. Numerous robust methods to generate gene libraries now exist. The limitation of this approach is the lack of high-throughput methods to identify the desired phenotype. With ₂₀^{x} variants possible for an x-amino acid protein, this search becomes an impossible task. General approaches that maybe used to identify the desired enzyme activity or property are: in vitro screening for activity, in vitro screening for binding, and in vivo selection for activity. The respective shortcoming of each is low throughput in the absence of automation, difficulty of linking binding to catalysis and difficulty in implementation for unnatural activity. Therefore, development of general high throughput methods to screen for the desired enzyme activity is critical for the advancement of organic synthesis using enzymes as catalysts.

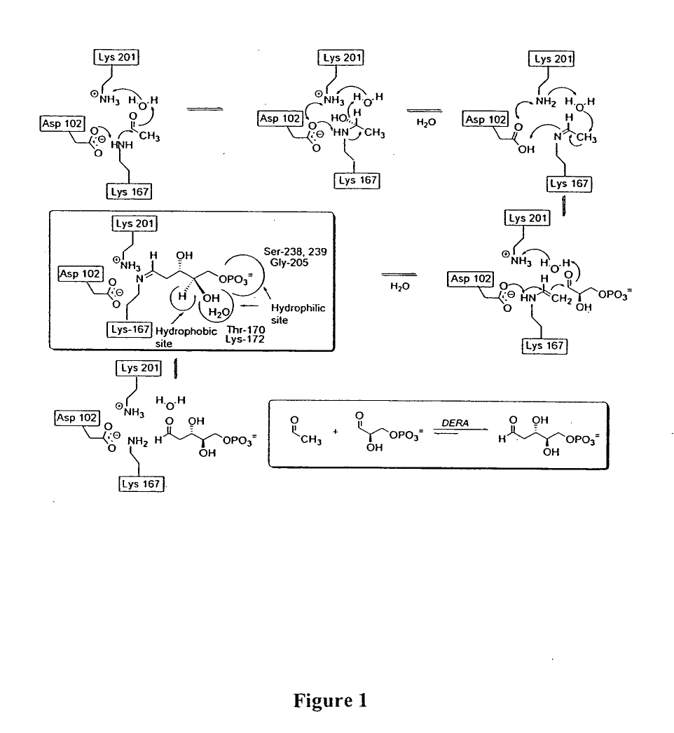

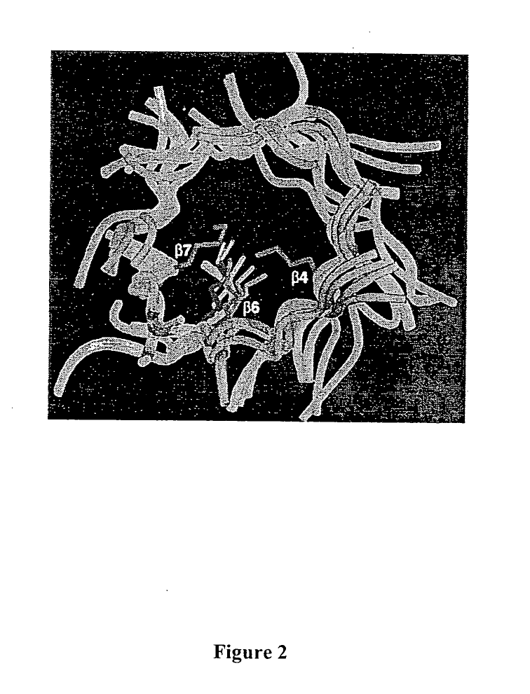

In the practice of the present invention, the X-ray structure of DERA and its proposed catalytic mechanism (FIG. 1) are used as a guide to design new nonphosphorylated substrates for the enzymatic reaction with inverted enantioselectivety and to alter the enzyme with mutagenesis to improve the turnover of the retroaldol reaction of the nonphosphorylated unnatural substrate D-2-deoxyribose (DR). Since the active site of DERA as well as most aldolases is a typical α/β barrel (FIG. 2), which has been shown to be a common scaffold (about 10% of known proteins have this fold) useful for alteration of the catalytic activity of other enzymes by directed evolution, it is thought to be a good model for development of novel DERA catalysts with expanded substrate specificity.

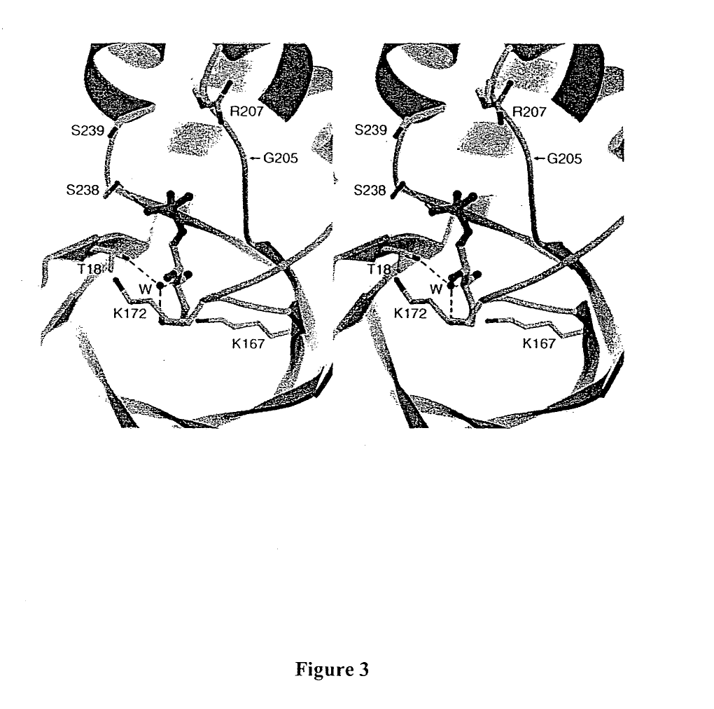

With the recently determined 1.05 ° three-dimensional structure of E. coli DERA in a carbinolamine covalent complex with bound DRP (FIG. 3), five variants were designed in hopes of improving activity for the unnatural substrate DR. The phosphate binding pocket is comprised of residues Gly 171, Lys172, Gly 204, Gly 205, Val206, Arg207, Gly 236, Ser238 and Ser239. However, only the side-chain of Ser238 forms a direct hydrogen bonding contact with the phosphate moiety of DRP.

The utility of a simple approach for changing substrate specificity by altering the electrostatic environment in an enzyme active site to one which is complementary to the electrostatic nature of the unnatural substrate has been demonstrated. Thus, by inspection of the enzyme active site, two basic residues were targeted for muta-genesis to acidic residues. The K172E and R207E variants were therefore prepared. In addition, three neutral side chains in the phosphate binding pocket were replaced with acidic ones, generating G205E, S238D and S239E variants. The goal of these designed mutations was to change the substrate specificity of WT-DERA from a preference for the negatively charged DRP to the nonphosphorylated, neutral DR substrate.

It was anticipated that an expanded substrate specificity of DERA in the retro-aldol direction would parallel an expanded substrate specificity in the aldol direction.

Accordingly, these variants are characterized in the retro-aldol direction. For each of the five variants, the activity with the natural substrate, DRP (Table 2) is substantially decreased as expected due to electrostatic repulsion between the introduced negatively charged residue and the negatively charged phosphate moiety of DRP.

TABLE 2
Effect of rationally designed DERA phosphate binding pocket mutations

| DETA mutant | Substrate | k_{cat} (s⁻¹) | K_{M} (mM) | k_{cat}/K_{M} (s⁻¹ mM⁻¹) | $\frac{[ {k}_{cat} \text{/} {K}_{M} ( DR ) ]}{[ {k}_{cat} \text{/} {K}_{M} ( DRP ) ]}$ | $\frac{[ {k}_{cat} \text{/} {K}_{M} ( mutant ) ]}{[ {k}_{cat} \text{/} {K}_{M} ( WT ) ]}$ |
|---|---|---|---|---|---|---|
| WT | DRP | 68 ± 1 | 0.64 ± 0.01 | 106 ± 2 | 1.9 × 10⁻⁵ | 1 |
| | DR | 0.107 ± 0.005 | 57 ± 7 | 0.0020 ± 0.0003 | | 1 |
| K172E | DRP | —^{a} | — | 0.0013 | 0.27 | 1.2 × 10⁻⁵ |
| | DR | 0.022 ± 0.02 | 63 ± 22 | 0.0003 ± 0.0001 | | 0.18 |
| G205E | DRP | — | — | 1.3 × 10⁻⁶ | 3.1 × 10⁻³ | 1.2 × 10⁻⁸ |
| | DR | — | — | 4.0 × 10⁻⁹ | | 2.0 × 10⁻⁶ |
| R207E | DRP | — | — | 0.0009 ± 0.0001 | 2.2 | 8.0 × 10⁻⁶ |
| | DR | 0.064 ± 0.001 | 33 ± 3 | 0.0019 ± 0.0002 | | 0.95 |
| S238D | DRP | 0.58 ± 0.05 | 61 ± 11 | 0.01 ± 0.001 | 0.13 | 9.0 × 10⁻⁵ |
| | DR | 0.21 ± 0.01 | 39 ± 6 | 0.005 ± 0.0009 | | 2.5 |
| S239E | DRP | 41 ± 1 | 4.3 ± 0.3 | 9.5 ± 0.7 | 2.7 × 10⁻⁴ | 0.09 |
| | DR | 0.175 ± 0.007 | 67 ± 8 | 0.0026 ± 0.0004 | | 1.3 |

^{a}No data.

In all cases, especially for R207E, the specificity for the unnatural substrate is improved as shown by the increase in the ratio of specificity constants for DR compared to DRP k_{cat}/K_{M} (DR)}/{k_{cat}/K_{M} (DRP)} of the variants versus WT. Clearly, this residue is critical to DRP transition state binding as evidenced by the data and is in agreement with the conserved nature of this residue for the nine closest homologues of E. coli DERA. However, for the shorter DR substrate, residue 207 may not be in sufficient proximity to effect a substantial change since, for this variant, DR specificity is virtually unchanged compared to WT. Two of the designed DERA variants exhibited higher than WT activity with DR as the substrate. Of these, the S238D variant is the most active, with a 2.5-fold improvement in k_{cat}/K_{M} compared to WT-DERA. S239E exhibits a 1.3-fold improvement in k_{cat}/K_{M} compared to WT. For both S239E and S238D, k_{cat}/K_{M} for the natural phosphorylated substrate is substantially decreased as would be expected due to electrostatic repulsion. Interestingly, in the WT structure only the side chain of S238 is in direct contact with the substrate and it seems that its proximity permits a degree of modulation of substrate specificity even for the smaller DR substrate. The G205E mutation yields a protein that is virtually inactive both with respect to DR and DRP. This residue is strictly conserved in the nine homologues of DERA and its mutation may effect a structural perturbation. The K172E mutation results in a 5-fold decrease in k_{cat}/K_{M} with the DR substrate.

In order to establish whether the improvement in the DERA catalyzed retro-aldol reaction is synthetically useful, we evaluate the efficiency of the DERA variants compared to WT to catalyze the aldol reaction between acetaldehyde and (±)-glyceraldehyde. In the aldol direction, the relative activity of the DERA variants as evaluated both by a spectrophotometric coupled-assay of substrate consumption and by thin layer chromatographic analysis of product formation is: S238˜DS239W>WT>R207E>K172E>G205E. The aldol reaction activity thus parallels the kinetic retro-aldol activity data and validates this approach. Therefore, two improved variants of DERA which catalyze both the aldol and retro-aldol reaction of a nonphosphorylated substrate have been developed.

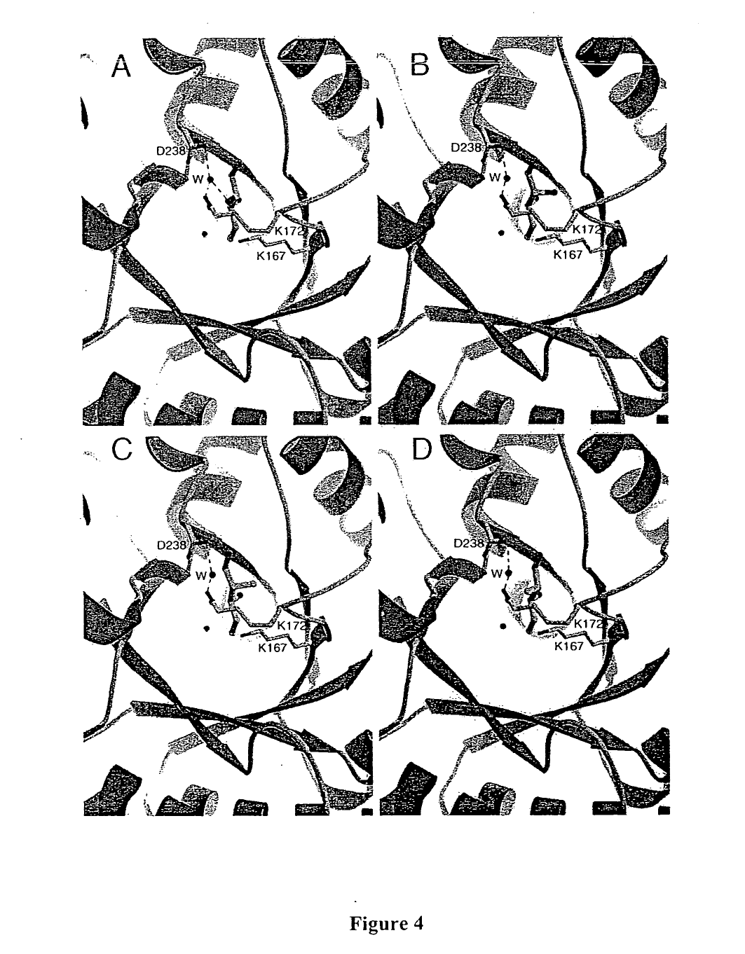

Molecular modeling (FIG. 4) shows that the terminal hydroxyl group of the product is able to form a 2.9-3.2 Å hydrogen bond to Asp238-CO₂. This may explain the increased activity of the S238D variant toward the nonphosphorylated substrate. Furthermore, optimization of the Asp side chain conformation (rotamer) results in the gain of a hydrogen bond (2.5 Å) to a water molecule in the active site. This water molecule forms a second hydrogen bond of 2.9 Å to the Nζ of Lys172. The first product complex (FIG. 4A) shows formation of a hydrogen bond (2.7 Å) between the hydroxyl group at the (R)-configured or D-configured C4 position with this water molecule. The hydrogen bond is absent in the second complex (FIG. 4B) with the (S)-configured C4 position and may explain the observed preference for the product formation shown in FIG. 4A.

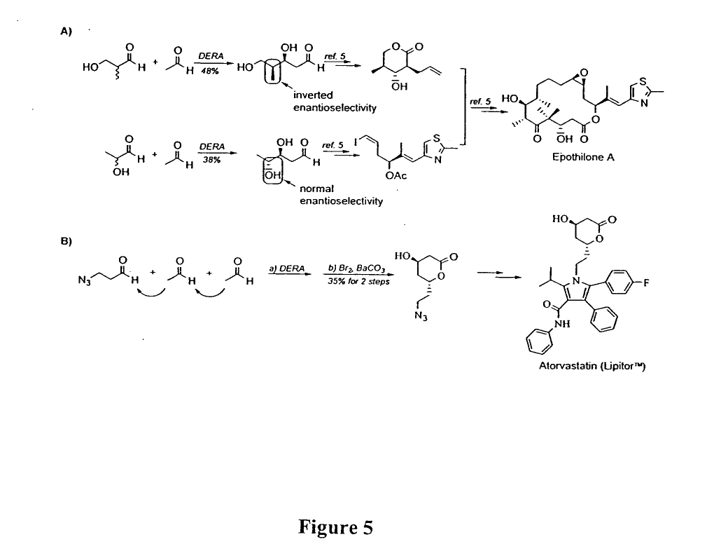

In addition to D-glyceraldehyde, DERA and the S238D variant accept other 2-substituted 3-hydroxy-propinaldehydes and inversion of enantioselectivity has been observed when 2-methyl- or 2-methoxy-3-hydrox-propinaldehyde is used as the substrate (FIG. 5). In both cases, the L-enantiomer is the preferred substrate for the wild type, but facial selectivity remains unchanged (FIG. 5A). This methyl-derived product has been used in the total synthesis of epothilones. In addition, the S238D variant accepts the L-2-methyl derivative as a better substrate with a 5-fold improvement in k_{cat}/K_{M} compared to that of the wild type. These results are consistent with structure-based molecular modeling. As described above and in FIG. 4, the water molecule interacting with the 2-hydroxy group of D-glyceraldehyde (corresponding to the 4-hydroxy group of the product) plays a key role in determining the enantioselectivity of DERA catalysis. The corresponding D-2-methyl derivative is not a substrate as the methyl group would be in close contact with the water molecule and the carbonyl oxygen of Thr170. On the other hand, binding of the 2-methyl group of the L-enantiomer to the enzyme is energetically more favorable (FIG. 4C), with the methyl group pointing to a more hydrophobic environment in van der Waals contact with Cα of Gly 171 (3.5 Å), Cβ of Ala203 (3.9 Å) and Cα of Gly 204 (3.6 Å). Both mechanistic and modeling studies thus reveal the important roles of the two water molecules in DERA catalysis: one is acting as acid and base in catalysis and the other is involved in the enantioselective binding of the acceptor substrate, as shown in FIG. 1.

While the S238D variant is in general better than the wild-type DERA to accept nonphosphor lated sub-strates as acceptors, it also catalyzes a novel sequential aldol reaction using 3-azidopropinaldehyde as the first acceptor and two molecules of acetaldehyde as donor to form an azidoethyl pyranose, a key intermediate useful for the synthesis of the cholesterol lowering agent Lipitor™ (FIG. 5B). The azidoaldehyde is, however, not a substrate for the wild-type enzyme.

While the 2.5-fold improvement in activity reported here is encouraging, considering that most mutations lead to decreases in activity, further enhancements are desirable. Though increasing the substrate scope of aldolases has previously been established by random mutagenesis, throughput limitations have allowed only a small percentage of the gene to be characterized. Thus, in order to rapidly evaluate the activity of a significant population of variants, a higher throughput activity-based screening methodology is essential. In preparation for a directed evolution program to identify DERA variants with expanded substrate scope, an in vivo selection system suitable for high-throughput analysis was therefore developed.

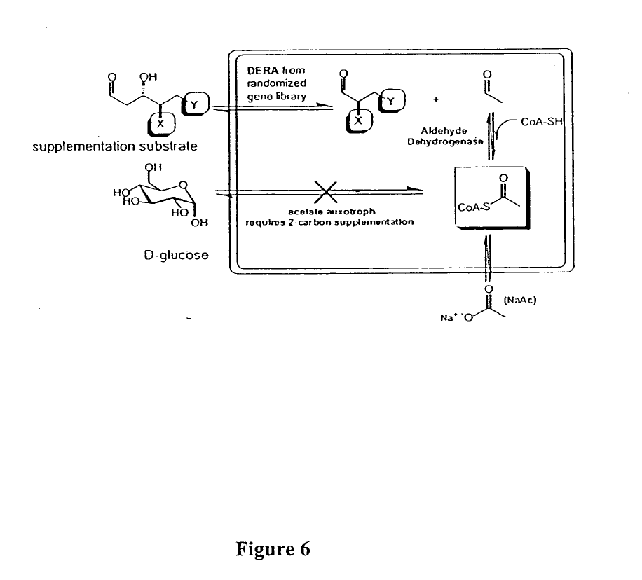

Having established the validity of screening for improved retro-aldol activity as indicative of the synthetic potential of the DERA, the retro-aldol direction was chosen for the development of a selection system. A cell that utilizes acetaldehyde as its sole carbon source or is dependent on acetaldehyde for growth was desired to aid selection of DERA variants with improved activity for DR or alternative unnatural substrates. SELECT (Δace, adhC, DE3), an E. coli strain that requires acetaldehyde for growth was engineered. Two features of SELECT are key. Firstly, the absence of a viable pyruvate dehydrogenase (aceF) affects an acetate auxotroph when grown in glucose as the sole carbon source. Secondly, the constitutive overproduction of an aerotolerant version of adhE, which has both alcohol dehydrogenase and acetaldehyde dehydrogenase activities, affects conversion of acetaldehyde to acetyl-CoA thus overcoming the acetate auxotroph (FIG. 6).

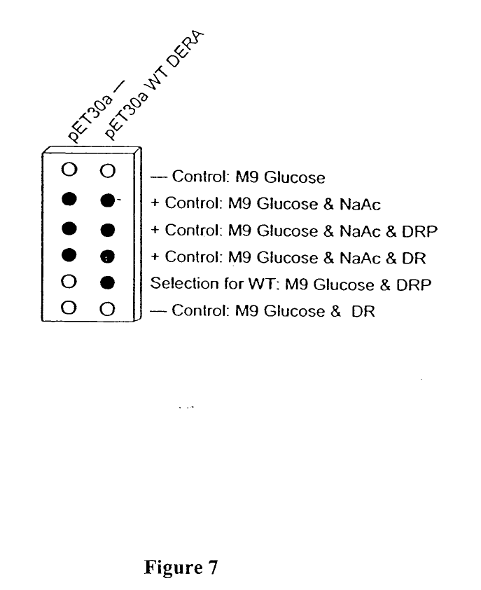

E. coli SELECT grows well in medium supplemented with either acetate or an acetaldehyde source and exhibits the desired phenotype (FIG. 7). SELECT was transformed with the DERA expressing plasmid, pET30a WT DERA, and growth conditions were then optimized for the expression of soluble active DERA enzyme. Selection conditions were further optimized using DERA's natural substrate DRP as a supplementation substrate and FIG. 6 illustrates that viable selection conditions are achieved. In the absence of 2-carbon supplementation, neither SELECT cells transformed with a plasmid which expresses WT DERA nor those transformed with a nonexpressing plasmid (−) grow. By contrast, both grow in the presence of sodium acetate supplementation. That both also grow in the presence of either sodium acetate together with DRP, or sodium acetate together with DR, demonstrates that neither of these supplementation substrates nor their metabolic products are toxic to the cells. Proof of principle for this selection system arises from the fact that only E. coli SELECT cells transformed with plasmid that expresses WT DERA grow when DRP is used as the supplementation substrate. Furthermore, that the endogenous genomic E. coli DERA is not expressed at a sufficiently high level to affect the use of DRP as a 2-carbon source by virtue of its metabolism to acetaldehyde and D-glyceraldehyde-3-phosphate was established. Since WT DERA cannot accept the unnatural substrate DR efficiently, neither of the SELECT cells transformed with nonexpressing plasmid (pET30a-) nor those transformed with DERA expressing plasmid (pET30a WT DERA) grow when supplemented with DR. A novel activity-based selection system is thus established and can be used to select for a DERA variant which can catalyze the retro-aldol reaction of DR and other nonphosphorylated substrates. Work is in progress to identify novel DERA variants for this purpose.

Several examples demonstrating the power of in vivo selection based methods for identifying variant enzymes which reverse the phenotype of a bacterial strain deficient in an enzyme with the desired activity have been reported. However, in most examples, such systems have been utilized to identify mutations which transform the activity of a natural enzyme into another natural enzyme to overcome auxotroph. In addition, several examples for which selection has been used to identify variants with native activity for an inactivated enzyme have also been demonstrated. To date, the reported examples of in vivo selection that have identified unnatural enzyme specificity or activity involve gene products which confer antibiotic resistance. However, more recently, an innovative growth selection based assay method for the identification of an error-prone T7 polymerase, and identification of a four-base codon tRNA were developed using an antibiotic resistance selection. Each of these elegant examples demonstrates the potential power a selection or complementation approach can have in identifying variants with improved or altered activity. Thus, the in vivo activity based selection system which utilizes the engineered E. coli strain SELECT to identify DERA variants with expanded substrate scope described here is one of the first examples of a selection method able to identify an enzyme with unnatural and synthetically useful substrate specificity in an ultra-high throughput manner.

Using the high-resolution X-ray structure of DERA and its catalytic mechanism, we have demonstrated that both the acceptor substrate and the enzyme can be changed to alter the efficiency and specificity of the enzymatic aldol reaction, including inversion of enantioselectivity using nonphosphorylated substrates and wild-type or S238D variants and new substrate specificity using the S238D variant. The S238D variant showed a 2.5-fold improvement in DERA activity with the unnatural substrate DR. It accepts 3-azidopropionaldehyde as a new substrate in a sequential aldol reaction to form a novel azidopyranose, while the wild-type enzyme is inactive toward this azidoaldehyde. To further improve the efficienc for identification of DERA variants to catal ze novel aldol reactions with nonphosphorylated substrates, we have developed a selection system which will be used to expand the acceptor specificity and stereoselectivity of this type of aldol reaction.

The invention will be further understood with reference to the following examples, which are purely exemplary, and should not be taken as limiting the true scope of the present invention as described in the claims.

### EXAMPLES

### Example 1

### Structure Based Mutagenesis to Expand Substrate Specificity of D-2-Deoxyribose-5-phosphate Aldolase

Nucleic acid manipulations were done according to standard procedures. TAQ DNA polymerase was from Stratagene. The Quiagen QIAprep Spin Miniprep Kit was utilized for plasmid preparation. PCR products were purified by electrophoresis on a 1% agarose gel and then extracted using the QIAEXII Agarose Gel Extraction Kit. Restriction endonucleases and T4 ligase were from New England Biolabs. Electrocompetent E. coli BL21 (DE3) cells, pET30 LIC and pET30a plasmids, and His-bind metal chelation resin were from Novagen. Oligonucleotide primers were prepared by Operon Technologies (San Diego, Calif.). DNA sequencing was performed at the Protein and Nucleic Acid Core Facility at The Scripps Research Institute on a ABI50 automated sequencer. UV kinetic assays were performed on a Cary 3 Bio UV-Vis spectrophotometer. Curve fitting was done by the non-linear least squares method using KaleidaGraph (Abelbeck Software). All reagents were purchased at highest commercial quality and used without further purification unless otherwise stated. Silica gel 60 (230-240 mesh) from Merck was used in chromatograph. High resolution mass spectra (HRMS) were recorded on IONSPEC-FTMS spectro-meter (MALDI) with DHB as matrix. ¹H NMR and ¹³C NMR spectra were performed on a Bruker AMX-500 instrument. IR spectra were recorded on a Perkin-Elmer 1600 series FT-IR spectrometer. Optical rotations were recorded on a Perkin-Elmer 241 polarimeter.

Cloning of WT DERA

The E. coli D-2-Deoxyribose-5-phosphate aldolase (DERA, EC 4.1.2.4) gene was PCR amplified from plasmid pVH17 (ATCC86963), using the forward primer 5′-ACCGATGACGACGACAAGGCCATGGCTATGACTGATCTGAAAG (SEQ ID NO: 1) and the reverse primer 5′-TGGTTGAGGAGAAGCCAAGCTTAGTAGCTGCTGGCGCT (SEQ ID NO: 2) and subcloned into the pET30 LIC vector (Novagen). E. coli D-2-Deoxyribose-5-phosphate aldolase (DERA, EC 4.1.2.4) gene was PCR amplified from the above construct, WT DERA pET30 LIC using the forward primer 5′-ACCGATGACGACGACAAGGCCATGGCTATGACTGATCTGAAAG (SEQ ID NO: 3) and the reverse primer 5′-TGGTTGAGGAGAAGCCAAG-CTTAGCTGCTGGCGCT (SEQ ID NO: 4) and then subcloned into the pET30a vector (Novagen) using the NcoI and HindIII restriction sites.

Site-Directed Mutagenesis

The following cloning primers were used: 5′-GACGACGACAAGATGCATATG (SEQ ID NO: 5), (forward) 5′-GAG-GAGAAGCCCGGTTTAGTA (SEQ ID NO: 6) (reverse). A 810-bp fragment was obtained by PCR using 20 mM of each of the dNTPs, 10 pM oligonucleotide primers, 10 ng template and 5 U Taq pol merase (Stratagene) in 100 mL DNA polymerase buffer. Mutagenesis primers used for double-sided overlap extension PCR were:
G207E, 5′ GCGGGCGGCGTGGAAACTGCGGAAGAT (SEQ ID NO: 7)
(forward), 5′-ACTTTCCGCAGTTTCCACGCCGCCCGC (SEQ ID NO: 8)
(reverse). S239E, 5′-TTTGGCGCTTCCGAACTGCTCGCAAGC (SEQ ID NO: 9)
(forward), 5′-GCTTGCCAGCAGTTCGGAAGCGCCAAA (SEQ ID NO: 10)
(reverse). S238D, 5′-CGCTTTGGCGCTGACAGCCTGCTGGCA. (SEQ ID NO: 11)
K172E, TCTA-CCGGTGAAGTGGCTGTG (SEQ ID NO: 12)
(forward), CACAGCCA-CTTCACCGGTAGA (SEQ ID NO: 13)
(reverse). G205E, 5′-AAACCGG-CGGGCGAAGTGCGTACTGCG (SEQ ID NO: 14)
(forward), 5/-CGCAGTACGCACTTCGCCCGCCGGTTT (SEQ ID NO: 15) (reverse). The mixture was thermocycled for 1 cycle at 94° C. for 5 min, then 30 cycles of {94° C. for 30 s, 55° C. for 30 s, 72° C. for 30 s} and then one cycle of 72° C. for 10 min.
   Protein Expression and Purification of DERA Variants

Plasmids were transformed into electrocompetent BL21 (DE3) and subjected to 1 h outgrowth at 37° C. in 1 mL SOC medium. These transformants (10-200 μL) were plated on LBₖₐₙ plates and incubated at 37° C. over-night. A starter culture was prepared by picking individual colony to inoculate a 100 mL Luria-Bertani (LB) starter culture containing 10 μg/mL kanamycin (kan) grown at 37° C., 220 rpm overnight. The starter culture was used to inoculate 1L LBₖₐₙ. Protein expression was induced at OD₆₀₀=0.6-0.8 by the addition of isopropyl-β-D-thiogalactoside (IPTG) to a final concentration of 0.5 mM. Cells were harvested 6 h after induction, by centrifugation at 4° C., 8000 rpm for 10 min and were stored at −78° C. The cell pellet was resuspended in 25 mL of 100 mM phosphate, 200 mM sodium chloride pH 7.5 chilled on ice. The cells were lysed by passing through a French press (SLM Instruments, Urbana, Ill.) compressed to 1500 psi and then released to ambient pressure, three times. Cell debris was pelleted by centrifugation at 4° C., 14,000 rpm for 1 h. The supernatant was filtered through a 0.2 μm cellular acetate membrane filter (Corning), and was loaded onto a Ni²⁺—NTA-agarose column with a bed volume of 2.5 mL pre-equilibrated with 100 mM phosphate, 200 mM sodium chloride, 5 mM imidazole, 5 mM β-mercaptoethanol pH 7.5 buffer. The column was washed with 40 mL of 100 mM phosphate, 200 mM sodium chloride, 20 mM imidazole, 5 mM β-mercaptoethanol, pH 7.5 buffer. Bound enzyme was then eluted with 20 mL of 100 mM phosphate, 200 mM sodium chloride, 20 mM imidazole, 5 mM β-mercaptoethanol pH 7.5 buffer, and was dialyzed against 50 mM triethanolamine hydrochloride pH 7.5 buffer at 4° C. Eluted enzymes were analyzed by SDS-PAGE and were found to be >95% pure in all cases. Enzyme solutions were aliquoted and frozen in liquid nitrogen and stored at −78° C. prior to use. Enzyme concentrations were determined by the Bradford procedure (Bio-Rad) using bovine serum albumin as a calibration standard.

DERA Cleavage (Retroaldol) Assay

Enzyme activity was monitored by the standard coupled assay using α-Glycerophosphate Dehydrogenase (α-GPD, EC 1.1.18), and Triosephosphate Isomerase (TPI, EC 5.3.1.1). Enzyme activity was assayed in the retro-aldol, decomposition direction with 0.01-4 mM D-2-deoxyribose-5-phosphate (DRP) or 5 to 200 mM D-2-deoxyribose in 50 mM triethanolamine hydrochloride pH 7.5 buffer using a GPD/TPI (1.6 U/mL Sigma G-1881) coupled enzyme system at 25° C. in the presence of 0.3 mM NADH by observing the rate of decrease of NADH concentration as monitored at 340 nm, ε=6220 M⁻cm⁻¹.

DERA Addition (Aldol) Assay

DERA enzyme activity was assayed in the aldol synthesis direction by determining the concentration of acetaldehyde remaining by a coupled endpoint assay with yeast alcohol dehydrogenase (YADH, EC 1.1.1.1). 200 mM acetaldehyde, which had been freshly distilled under anerobic conditions, 200 mM (±)-glyceraldehyde and 0.2 mg/mL DERA in 50 mM triethanolamine, pH 7.5 buffer which had been deoxygenated with N₂, were incubated under an N₂ atmosphere at 22° C. At various time points, 50 μL aliquots were withdrawn and quenched into 15 μL of 60% perchloric acid. After a 5 min incubation on ice, 890 μL 1 M triethanolamine, pH 7.5 buffer and 45 μL 4 N NaOH were added to neutralize the solution. 20 μL of this solution was then assayed for remaining acetaldehyde. The amount of acetaldehyde remaining was equated to moles NADH consumed, as determined in triethanolamine pH 7.5 buffer containing 0.3 mM NADH, 20 μL the above quenched reaction aliquot and 0.05 mg/mL YADH. DR product formation was also confirmed by silica gel TLC with ethylacetate running solvent and p-anisaldehyde developing stain. R_{f}: glyceraldehyde=0.04 (stains brown) R_{f}:2-deoxyribose=0.1 (stains blue).

Construction of E. coli SELECT Strain

First, DC81 was transduced with P1 grown on JC1552 (aceF+leu−) and transductants able to grow without acetate were selected in the presence of leucine. DC119 was one such aceF+ transductant, which also received the leu mutation from JC1552 and hence required leucine. Next, DC119 was transduced with P1 grown on DC34 (ΔaceEF leu+) and transductants able to grow without leucine were selected on minimal medium E containing succinate (0.4%) plus acetate (0.2%) as carbon source. Transductants were screened for those unable to grow on succinate alone, that is, those receiving succinate (0.4%) plus acetate (0.2%) as the carbon source. Transductants were screened for those unable to grow on acetate alone, that is, those receiving the Δ(aroP-aceEF) 15 deletion and therefore requiring exogenous acetate. DC489 was one such transductant. E. coli strain SELECT was then prepared by generating the λDE3 lysogen of DC489 using the Novagen λDE3 lysogenization kit (69734-3) according to manufacturer's directions. E. coli strains DC81, DC34, and JC1552 were used for construction of SELECT.

Development of Liquid Selection Conditions

Plasmids were transformed into electrocompetent SELECT cells and subjected to 1 h outgrowth at 37° C. in 1 mL SOC medium supplemented with 0.1% sodium acetate. The cells were then collected by centrifugation at 4° C., 3000 rpm for 10 min. The supernatant was discarded and the pellet gently resuspended M9 0.2% glucose. This was repeated twice. The cells were then diluted to OD₆₀₀=0.001 in M9 0.2% glucose, 0.01 mM IPTG, 10 μg/mL kanamycin. The appropriation supplementation substrate (sodium acetate, D-2-deoxyribose-phosphate or D-2-deoxyribose) was then added at 0.1% w/w concentration. After an appropriate selection time at 37° C., typically 24-72 h, the cells were harvested by centrifugation and their amplified plasmids isolated.

Molecular Modeling

The DERA enzyme S238D mutation was generated using the program O¹ and the side chain placed in a common rotamer position. The product molecules displayed in FIG. 5A-D were generated using the Builder module of InsightII (2000) (Accelr s Inc.) and energy minimized. They were manually placed in the enzyme active site based on the existing Schiff base crystal structure (PDB code 1JCJ ). Hydrogen atoms on the protein residues and on relevant water oxygen atoms were added using the Biopolmer module. For energy minimization, the CVFF force field was used. All minimizations were carried out with the Discover module using a distant dependent dielectric constant. In the first round of minimization all non-hydrogen atoms were constrained to fixed positions, and steepest descent and conjugate gradient energy minimizations were performed for 100 iterations each. Thereafter, constraints for the product molecule and Asp238 were released and the minimization procedure was repeated.

Sequential Asymmetric Aldol Reaction

To a mixture containing 3-azidopropinaldehyde (600 mg, 6.0 mmol) was added a buffer solution (36 mL, pH=7.5), which contained variant S238D DERA (about 200 U based on the assay using DRP as substrate). The resulting solution was stirred in the dark for 6 days under argon. The reaction was quenched with 2 volumes of acetone. The mixture was then stirred at 0° C. for 1 h and centrifuged to remove the precipitated enzyme. The aqueous phase was concentrated in vacuo, and the residue was passed through a short silica column eluted with EtOAc. The elutant was concentrated and afforded the crude product (560 mg, 3.0 mmol).

To a mixture of the lactol above (560 mg, 3.0 mmol) and BaCO₃ (0.8 g, 4.0 mmol) in H₂O (20 mL) at 0° C. was added slowly freshly opened Br₂ (180 μL, 3.4 mmol). The mixture was stirred in the dark overnight. After filtration, water was removed in vacuo. Purification of the residue by flash chromatography (silica, 1:1 hexane/EtOAc) afforded the product (391 mg, 35% for 2 steps). [α]D=72.0° (c=1.0, CHCl₃); IR (film): 3421.1, 2928.0, 2102.8, 1718.2, 1254.8, 1072.2 cm⁻¹; ¹H NMR (500 MHz, CDCl₃) δ 4.85 (m, 1H), 4.40 (m, 1H), 3.54 (dd, J=5.8, 7.3 Hz, 2H), 2.76 (br. s, 1H), 2.67 (m, 2H), 2.00 (br. d, J=14.3 Hz, 1H), 1.95 (m, 1H), 1.87 (m, 1H), 1.77 (m, 1H); ¹³C NMR (125 MHz, CDCl₃ ) δ 170.30, 72.86, 62.37, 47.06, 38.45, 35.72, 34.73; HRMS m/e calcd for (M⁺)C₇H₁₁N₃O₃: 185.0800; found: 208.0693 (M+Na).

### Example 2

### Aldolase-Catalyzed Asymmeteric Synthesis of Novel Pyranose Synthons

General Methods

All reactions were carried out under an argon atmosphere with dry, freshly distilled solvents under anhydrous conditions, unless otherwise noted Tetrahydrofuran (THF) and diethyl ether were distilled from sodium-benzophenone, and dichloromethane (CH₂Cl₂) and toluene from calcium hydride. All reagents were purchased at highest commercial quality and used without further purification unless otherwise stated. Silica gel 60 (230-240 mesh) from Merck was used in chromatography. High resolution mass spectra (HRMS) were recorded on a VG ZAB-ZSE instrument under fast atom bombardment (FAB) conditions with NBA as the matrix or IONSPEC-FTMS spectrometer (MALDI) with DHB as matrix. ¹H NMR spectra and ¹³C NMR were performed on a Bruker AMX-500. or AMX-600 instruments. IR spectra were recorded on a Perkin-Elmer 1600 series FT-IR spectrometer. Optical rotations were recorded on a Perkin-Elmer 241 polarimeter. General enzymatic reactions catalyzed by DERA: To a 100 ml buffer solution (0.1M KH₂PO₄, pH=7.5) containing 0.1M acceptor aldehyde and 0.3M donor (acetaldehyde or acetone) was added 3000 units of DERA. The resulting solution was stirred in the dark for 3-6 days under argon. The reaction was quenched by addition of 2 volumes of acetone. The mixture was then stirred at 0° C. for 1 hour and centrifuged to remove the precipitated enzyme. The aqueous phase was concentrated in vacuo, and the residue was purified by flash chromatography (silica, 1:2 to 4:1 EtOAc:hexane).

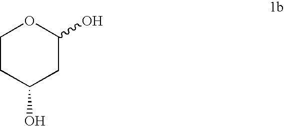

Yield: 65%; [α]_{D}=−19.0° (c=0.5, CH₃OH); IR (film): 3360.5, 2931.0, 1119.9, 1055.2;

¹H NMR (600 MHz, CDCl₃) δ major isomer: 5.09 (s, 1H), 4.21 (m, 1H), 4.11 (br. s, 1H), 3.56 (dt, J=4.8, 11.9 Hz, 1H), 2.79 (s, 1H); minor isomer: 5.33 (s, 1H), (4.06 (br. s, 1H), 3.95 (dt, J 3.1, 12.7 Hz, 1H), 3.79 (dt, J=4.4, 12.0 Hz, 1H), 3.01 (s, 1H), 2.05-1.55 (m, 8H); ¹³C NMR (150 MHz, CDCl₃) δ major isomer: 93.10, 65.03, 59.15, 37.62, 32.95; minor isomer: 92.58, 63.77, 56.40, 39.70, 34.47; HRMS m/e calcd. for (M⁺) C₅H₁₁O₃: 118.0630; found: 141.0523 (M+Na).

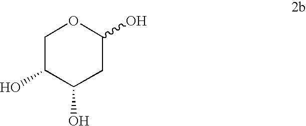

Yield: 60%; the ¹H NMR spectrum is consistent with the published data. [R. U. Lemieux, Carbohydr. Res. 1971. 20, 59]

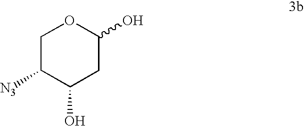

Yield: 47%; IR (film): 3383.8, 2907.5, 2104.9, 1266.8, 1072.9; ¹H NMR (500 MHz, CDCl₃) δ major isomer: 5.14 (dt, J=2.6, 7.7 Hz, 1H), 4.22 (m, 1H), 4.13 (dd, J=10.1, 11.9 Hz, 1H), 3.71 (dd, J=4.8, 11.8 Hz, 1H), 3.58 (ddd, J=2.9, 4.8,.9.9 Hz, 1H), 2.93 (d, J=52 Hz, 1H), 2.10 (ddd, J=2.6, 10.2, 19.3 Hz, 1H), 1.92 (dt, J=3.3, 16.8 Hz); minor isomer: 5.32 (q, J=3.3 Hz, 1H), 4.24 (m, 1H), 4.10 (dd, J=2.6, 12.5 Hz, 1H), 3.84 (dd, J=4.8, 12.1 Hz, 1H), 3.77 (q, J=3.3 Hz, 1H), 1.98 (ddd, J=3.0,9.9, 12.8 Hz), 1.88 (dt, J=4.0, 13.2 Hz, 1H); ¹³C NMR (125 MHz, CDCl₃) δ major isomer: 92.28, 67.12,58.97, 56.83, 35.14 minor isomer: 91.74, 64-92, 61.07, 60.63, 35.33.

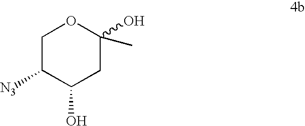

Yield: 28%;.IR (film): 3365.8, 2931.0, 2096.6, 1707.5, 1266.8, 1084.6; ¹H NMR (600 MHz, CD₃OD) δ major isomer: 4.09 (m, 1H), 3.93 (dd, J=1.8, 7.8 Hz, 1H), 3.58 (m, 2H), 1.73 (dd, J=4.8, 12.5 Hz, 1H), 1.65 (t, J=12.5 Hz, 1H), 1.29 (s, 3H); open chain form: 4.00 (m, 1H), 3.72 (dd, J=4.0, 11.7 Hz, 1H), 3.51 (dd, J=7.7, 11.7 Hz, 1H), 3.54 (m, 1H), 2.59 (dd of AB, J=1.8, 9.6 Hz, 1H), 2.57 (dd of AB, J=5.2, 9.6 Hz, 1H), 1.92 (s, 3H); ¹³C NMR (150 MHz, CD₃OD) δ major isomer: 97.70, 67.21, 62.87, 39.85, 29.45; open chain form: 209.76, 69.38, 68.41, 62.55, 47.80, 30.72; HRMS m/e calcd. for (M⁺) C₆H₁₁N₃O₃: 173.0800; found: 196.0702 (M+Na).

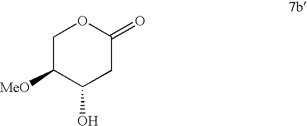

7b yield: 22%, characterized by its lactone form 7b′: IR (film): 3459.8, 2931.0, 1719.2, 1249.1, 1096.4; ¹H NMR (600 MHz, CD₃OD) δ 4.55 (dd, J=3.1, 12.9 Hz, 1H), 4.29{(dd, J=4.4, 12.2 Hz, 1H), 4.21 (m, 1H), 3.47 (s, 3H), 3.46 (m, 1H), 2.97 (dd, J=4.8, 11.5 Hz, 1H), 2.57 (dd, J=4.8, 17.9 Hz, 1H), 2.24 (s, 1H); ¹³C NMR (150 MHz, CD₃OD) δ 169.04, 76.24, 66.30, 66.19, 57.27, 35.87; ESI calcd. for C₆H₁₀O₄: 146; found: 169 (M+Na).

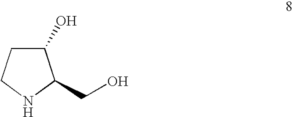

Preparation of hydropyrrolidine 8: To a solution of 3b (57 mg, 0.36 mmol) in 10 ml methanol was added 5 mg Pd/C. The mixture was hydrogenated under 50 psi H₂ overnight. After filtration through Celite, the mixture was concentrated in vacuo. The residue was purified by flash chromatography (silica, 2:1 EtOAc:hexane) to afford 8 (35 mg, 85%): [α]_{D}=42.6° (c=0.5, CH₃OH); IR (film): 3354.2, 2931.0, 1413.7, 1121.9; ¹H NMR (500 MHz, CD₃OD) δ 4.05 (dt, J=3.7, 7.3 Hz, 1H), 3.55 (dd, J=4.8, 11.4 Hz, 1H), 3.50 (dd, J=6.2, 11.8 Hz, 1H), 3.00 (m, 3H), 1.93 (m, 1H), 1.70 (m, 1H); ¹³C NMR (125 MHz, CD₃OD) δ 73.82, 69.14, 62.35, 45.18, 35.21; HRMS m/e calcd. for (M⁺) C₅H₁₁NO₂: 117.0790; found: 118.0863 (M+H).

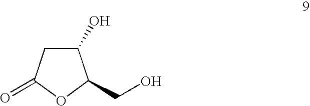

Preparation of lactone 9: To a mixture of 2b (60 mg, 0.44mmol) and BaCO₃(140 mg, 0.71 mmol) in H₂O (6.0 ml) at 0° C. was added slowly freshly opened Br₂ (30 μl, 0.57 mmol). The resulting mixture was stirred in dark overnight. After filtration, water was removed in vacuo. Purification of the residue by flash chromatography (silica, 2:1 EtOAc:hexane) to afford 9 (44 mg, 75%) as a clear oil: [α]_{D}=3.1° (c=2.9, CH₃OH); IR (film): 3384.2, 1773.2, 1189.3, 1073.4, 609.2; ¹H NMR (600 MHz, CD₃OD) δ 4.36 (dt, J=2.2, 6.5 Hz, 1H), 4.30 (m, 1H), 3.70 (dd, J=3.5, 12.2 Hz, 1H), 3.62 (dd, J=3.5, 12.7 Hz), 2.84 (dd, J=7.0, 17.9 Hz, 1H), 2.30 (dd, J=2.6, 18.0 Hz, 1H); ¹³C NMR (150 MHz, CD₃OD) δ 178.66, 90.14, 69.67, 62.50, 39.13; HRMS m/e calcd. for (M⁺) C₅H₈O₄: 132.0422; found: 155.0310 (M+Na).

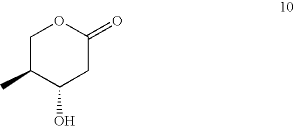

Preparation of lactone 10: To a mixture of lactol 5b (14.5 g, 0.11 mol) and BaCO₃ (30 g, 0.15 mol) in H₂O (600 ml) at 0° C. was added slowly freshly opened Br₂ (5.8 ml, 0.11 mol). The resulting mixture was stirred in dark overnight. After filtration, water was removed in vacuo. Purification of the residue by flash chromatography (silica, 2:1 EtOAc:hexane) to afford lactone 10 (7.9 g, 62%) as a clear oil: [α]_{D}=37.9° (c=0.24, CHCl₃); IR (film): 3398.0, 2966.3, 2919.3, 1724.3 1231.5, 1043.5 cm⁻¹; ¹H NMR (500 MHz, CDCl₃) δ 4.41 (dd, J=4.7, 11.4 Hz, 1H), 3.87 (dd, J=9.1, 11.4 Hz, 1H), 3.82 (m, 1H), 2.94 (dd, J=5.9, 17.6 Hz, 1H), 2.51 (dd, J=7.4, 17.6 Hz, 1H), 2.19 (d, J=4.4 Hz, 1H), 1.96 (m, 1H), 1.09 (d, J=6.6 Hz, 3H); ¹³C NMR (125 MHz, CDCl₃) δ 170.19, 70.96, 69.38, 38.42, 35.92, 13.27. ESI calcd. for (M⁺) C₆H₁₀O₃: 130; found: 153 (M+Na).

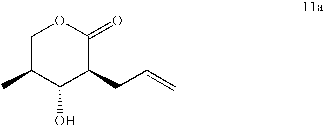

Preparation of 11a: To a stirred solution of diisopropylamine (4.7 ml, 33.5 mmol) in anhydrous THF (50 ml) was added n-butyllithium (21.1 ml, 1.6N in hexane, 33.7 mmol) at 0° C. The mixture was stirred for 20 min and then cooled to −78° C., a solution of 10 (1.88 g, 14.5 mmol) in THF (50 ml, washed with 2×10 ml THF) and HMPA (14.1 ml) was added slowly. The solution was stirred at 2 h and then the second potion of n-butyllitium (17.6 ml, 28.2 mmol) was added and the resulted mixture was stirred for another 30 min. Freshly distilled allyl bromide (6.5 ml, 75 mmol) was added slowly. The reaction mixture color changed from clear to green, brown, black and finally changed back to clear. After 36 h, AcOH (3.8 ml, 66 mmol) was added to quench the reaction. Water (100 ml) was then added. After most THF was removed, the mixture was extracted with CH₂Cl₂ (4×150 ml). The combined organic layer was dried over Na₂SO₄, and concentrated in vacuo. The residue was purified by flash chromatography (silica, 3:1 hexane: EtOAc) to give the alkylated lactone 11a (1.88 g, 85%). 0.22 g (12%) starting lactone was recovered. [α]_{D}=25.0° (c=0.32, CHCl₃); IR (film): 3405.3, 2964.2, 2902.7, 1731.5, 1635.9, 1400.0, 1312.8, 1205.1, 1041.0, 1000.0, 928.0; ¹H NMR (500 MHz, CDCl₃) δ 5.87 (m, 1H), 5.17 (m, 2H), 4.28 (dd, J=4.4, 11.4 Hz, 1H), 3.81 (dd, J=10.3, 11.4 Hz, 1H), 3.50 (td, J=4.8, 9.2 Hz, 1H), 2.70 (t, J=5.9 Hz, 2H), 2.56 (dt, J=55,9.2 Hz, 1H), 2.09 (d, J=4.4 Hz, 1H), 2.03(m, 1H), 1.06 (d, J=6.6 Hz, 3H); ¹³C NMR (125 MHz, CDCl₃) δ 172.16, 135.13, 118.49, 73.06, 70.43, 48.99, 36.21, 32.98, 13.29; HRMS m/e calcd. for (M⁺) C₉H₁₄O₃: 170.0973; found: 171.1017 (M+H).

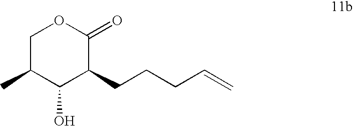

Yield: 32%, 48% recovery; ¹H NMR (600 MHz, CDCl₃) δ 5.82 (m, 1H), 5.00 (m, 2H), 4.28 (dd, J=4.4, 11.4 Hz, 1H), 3.81 (dd, J=9.9, 11.4 Hz, 1H), 3.44 (dt, J=4.0, 8.8 Hz, 1H), 2.46 (dt, J=5.2, 10.1 Hz, 1H), 2.20-1.45 (m, 7H), 1-07 (d, J=7.0 Hz, 3H); ¹³C NMR (150 MHz, CDCl₃) δ 173.01, 138.28, 114.85, 73.44, 70.22, 49.30, 36.76, 33.76, 27.85, 25.87, 13.48; ESI calcd. for C₁₁H₁₈O₃: 198; found: 233 (M+Cl).

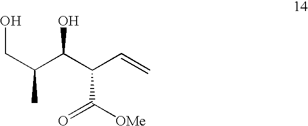

Preparation of diol acid 14: To the solution of 11a (1.0 g, 6.5 mmol) in 200 ml dry methanol was added MeONa (30 ml 25%, 13 mmol) at −35° C. The mixture was stirred at −30° C. for 15 h. After the pH was adjusted to 7.0 with Dowex (H⁺ form) and filtration, the methanol was evaporated. Purification of the residue by flash chromatography (silica, 4:1 hexane: EtOAc) afforded 14 (0.79, 60%) as a clear oil and starting material (0.14 g, 14%). [α]_{D}=8.4° (c=0.38, CHCl₃); IR (film): 3394.9, 2943.6, 1717.9, 1642.6, 1435.9, 1194.9, 1117.9, 1025.8, 984.6; ¹H NMR (500 MHz, CDCl₃) δ 5.80 (m, 1H), 5.04 (m, 2H), 4.03 (ddd, J=2.6, 4.0, 8.8 Hz, 1H). 3.77 (m, 1H), 3.69 (m, 1H), 3.67 (s, 3H), 2.75 (d, J=4.4 Hz, 1H), 2.70 (td, J=4.4, 9.9 Hz, 1H), 2.60 (m, 1H), 2.41 (m, 1H), 1.85 (t, J=4.8 Hz, 1H), 1.68 (m, 1H), 1.01 (d, J=6.9 Hz, 3H); ¹³C NMR (125 MHz, CDCl₃) δ 174.38, 135.36, 116.95, 74.05, 67.51, 51.56, 49.56, 37.43, 33.58, 9.60; ESI: calcd. for (M⁺) C₁₀H₁₈O₄:202; found: 225 (M+Na)

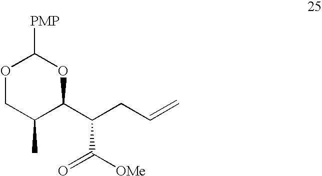

Preparation of 25: To a mixture of 14 (195 mg, 0.97 mmol) and PMB dimethyl acetal (0.6 ml, 2.5 mmol) in 3 ml dry DMF was added camphor sulfonic acid (7 mg) at 0° C. The reaction solution was stirred overnight and quenched with 0.2 ml sat. NaHCO₃ solution. The solvent was removed in vacuo. The residue was purified by flash chromatography (silica, toluene) to give methyl ester 25 (294 mg, 95%) [α]_{D}=−9.8° (c=0.94, CHCl₃); IR (film): 2959.2, 1730.7, 1610.1, 1514.6, 1393.9, 1248.2, 1112.5, 1032.0, 828.0; ¹H NMR (500 MHz, CDCl₃) δ 7.41 (d, J=9.0 Hz, 2H), 6.89 (d, J=9.0 Hz, 2H), 5.74 (m, 1H), 5.46 (s, 1H), 5.04 (m, 2H), 4.06 (dd, J=2.2, 11.4 Hz, 1H), 4.03 (dd, J=2.2, 10.3 Hz, 1H), 3.98 (dd, J=1.5, 11.0 Hz, 1H), 3.80 (s, 3H), 3.68 (s, 3H), 2.78 (dt, J=3.7, 9.9 Hz, 1H), 2.66 (m, 1H), 2.36 (dt, J=9.2, 14.0 Hz, 1H), 1.59 (m, 1H), 1.20 (d, J=7.0 Hz, 3H); ¹³C NMR (125 MHz, CDCl₃) δ 173.25, 159.94, 134.85, 131.14, 127.26, 117.04, 113.60, 101.88, 79.35, 73.55, 55.29, 51.52, 48.06, 33.63, 30.57, 11.32; HRMS m/e calcd. for (M⁺) C₁₈H₂₄O₅ 320.1624, found: 343. 1520 (M+Na).

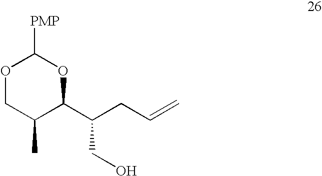

Preparation of 26: To a suspension of LiAlH, (550 mg, 95%, 14 mmol) in dry ether (130 ml) was slowly added a solution of 25 (1.27 g, 3.70 mmol) in 20 ml (washed with 10 ml+10 ml) ether at 0° C. The mixture was stirred for 2 h at room temperature and quenched with 1 ml water and 2 ml 1N NaOH. The mixture was diluted with 100 ml ether and extracted with ether (3×200 ml). The combined organic layer was washed with brine, dried (Na₂SO₄), filtered and concentrated in vacuo. The residue was purified by flash chromatography (silica, 3:2 hexane:EtOAc) to give 26 (0.98g, 90%) as a clear oil. [α]_{D}=−34.9° (c=0.43, CHCl₃); IR (film): 3418.3, 2966.6, 2921.2, 2853.6, 1608.0, 1393.5, 1246.6, 1105.5, 1032; ¹H NMR (500 MHz, CDCl₃) δ 7.50 (d, J=8.5 Hz, 2H), 6.97 (d, J=8.5 Hz, 2H), 5.94 (m, 1H), 5.55 (s, 1H), 5.19 (m, 2H), 4.17 (dd, J=2.2, 11.0 Hz, 1H), 4.10 (dd, J=1.5, 11.2 Hz, 1H), 3.97 (dd, J=2.2, 9.9 Hz, 1H), 3.88 (s, 3H), 3.81 (m, 1H), 3.71 (m, 1H), 2.62 (br. d, J=12.4 Hz, 1H), 2.32 (dt, J=8.8, 13.9 Hz, 1H), 1.93 (m, 1H), 1.84 (m, 1H), 1.46 (m, 1H), 1.28 (d, J=7.0 Hz, 3H); ¹³C NMR (125 MHz, CDCl₃) δ 160.24, 137.54, 131.90, 127.66, 117.16, 113.99, 102.31, 80.08, 74.29, 61.19, 55.72, 41.76, 32.24, 30.65, 11.94; HRMS m/e calcd. for (M⁺) C₁₇H₂₄O₄: 292.1674; found: 315.1567 (M +Na).

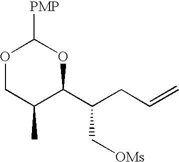

Preparation of 27: Methanesulfonyl chloride (0.5 ml, 6.5 mmol) was added slowly to a stirred solution of 26 (0.95 g, 3.2 mmol) in anhydrous CH₂Cl₂ (100 ml) containing triethylamine (1.2 ml, 8.4 mmol) under argon at 0° C. The solution was stirred at room temperature overnight and quenched with 50 ml saturated NaHCO₃ solution. The mixture was then extracted with CH₂Cl₂ (3×200 ml). The organic layer was washed with brine and concentrated in vacuo. The residue was purified by flash chromatography (silica, 3:2 hexane:EtOAc) to give mesylated compound 27 (1.13, 94%). [α]_{D}=−21.20 (c=0.92, CHCl₃); IR (film): 2965.8, 2932.9, 2858.7, 1613.8, 1515.0, 1399.7, 1354.4, 1247.3, 1169.0, 1111.4, 1033.1 cm⁻¹; ¹H NMR (600 MHz, CDCl₃) δ 7.38 (d, J=8.8 Hz, 2H), 6.86 (d, J=8.8 Hz, 2H), 5.74 (m, 1H), 5.43 (s, 1H), 5.10 (m, 2H), 4.21 (dd of AB, J=3.5, 9.5 Hz, 1H), 4.19 (dd of AB, J=3.5, 9.5 Hz, 1H), 4.05 (d of AB, J=10.9Hz, 1H), 4.01 (d of AB, J=10.9 Hz, 1H), 3.83 (d, J=10.1 Hz, 1H), 3.78 (s, 3H), 2.99 (s, 3H), 2.58 (br. d, J=14.2 Hz, 1H), 2.16 (dt, J=9.2, 18.8 Hz, 1H), 2.00 (ddd, J=3.5, 7.0, 16.6 Hz, 1H), 1.73 (br. d, J=6.6 Hz, 1H), 1.18 (d, J=6.6 Hz, 3H); ¹³C NMR (150 MHz, CDCl₃) δ 159.96, 135.39, 131.19, 127.24, 118.00, 113.62, 101.95, 78.95, 73.61, 67.24, 55.30, 39.23, 37.15, 31.09, 29.98, 11.37; HRMS m/e calcd. for (M⁺) C₁₈H₂₆O₆S: 370.1450; found: 393.1344(M+Na).

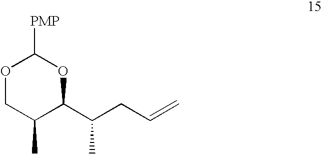

Preparation of 15: A solution of above compound 27 (635 mg, 1.71 mmol) in 30 ml ether was treated LiAlH₄(391 mg, 95%, 10 mmol) at 0° C. The suspension was stirred for 2 h at room temperature and quenched with water (1 ml) and 1N NaOH (2 ml). The resulting mixture was stirred for another 30 min and water (20 ml) was added. It was extracted with ether (3×50 ml). The organic layer was washed with brine and concentrated in vacuo. The residue was purified by flash chromatography (silica, 4:1 hexane:EtOAc) to give 15 (416 mg, 88%) as a clear oil. [α]_{D}=−22.4° (c=0.46, CHCl₃); IR (film): 2954.3, 2919.2 2837.0, 1607.6, 1513.6, 1460.7, 1384.3, 1243.3, 1161.0, 1114.0, 1031.7, 996.5, 826.1 cm⁻¹; ¹H NMR (500 MHz, CDCl₃) δ 7.43 (d, J=8.8 Hz, 2H), 6.88 (d, J=8.5 Hz, 2H), 5.79 (m, 1H), 5.43 (s, 1H), 5.03 (m, 2H), 4.05 (dd of AB, J=2.2, 11.4 Hz, 1H), 4.02 (dd, of AB, J=1.9, 11.4 Hz, 1H), 3.80 (s, 3H), 3.49 (dd, J=2.2, 10.2 Hz, 1H), 2.50 (br. d, J=13.6 Hz, 1H), 1.93 (dt, J=8.5, 13.6 Hz, 1H), 1.77 (m, 1H), 1.67 (m, 1H), 1.16 (d, J=7.0 Hz, 3H), 0.82 (d, J=6.6 Hz, 3H); ¹³C NMR (125 MHz, CDCl₃) 5 159.75, 136.81, 131.60, 127.21, 116.35, 113.54, 101.55, 83.24, 73.96, 55.29, 36.94, 33.95, 29.94, 13.70, 10.95; ESI calcd. for (M⁺) C₁₇H₂₄O₃: 276; found: 277 (M+H).

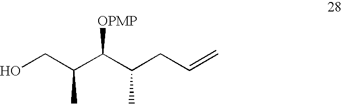

Preparation of 28: To a solution of 15 (201 mg, 0.73 mmol) in 10 ml toluene at 0° C. was added 0.7 ml DIBAL (1.5M, 1.05 mmol). The mixture was stirred overnight at room temperature. The reaction was then quenched with water and extracted with EtOAc (4×30 ml). The organic layer was washed with brine and concentrated in vacuo. Purification of the residue by flash chromatography (silica, 7:3 hexane:EtOAc) afforded 28 (187 mg. 93%). [α]_{D}=1.0° (c=0.7, CHCl₃); IR (film): 3401.1, 2966.2, 2919.2, 2876.2, 2353.4, 2328.7, 1610.5, 1510.6, 1457.7, 1381.4, 1243.3, 1025.9, 814.3 cm⁻¹; ¹H NMR (500 MHz, CDCl₃) δ 7.25 (d, J=8.8 Hz, 2H), 6.85 (d, J=8.8 Hz, 2H), 5.77 (m, 1H), 5.00 (m, 2H), 4.54 (d, J=11.0 Hz, 1H), 4.48 (d, J=11.0 Hz, 1H), 3.78 (s, 3H), 3.57 (m, 2H), 3.33 (dd, J=3.0, 7.7 Hz, 1H), 2.45 (dtd, J=1.8,3.7, 13.6 Hz, 1H), 1.98-1.90 (m, 2H), 1.88-1.80 (m, 1H), 0.91 (d, J=7.0 Hz, 3H), 0.88 (d, J=6.6 Hz, 3H); ¹³C NMR (125 MHz, CDCl₃) δ 159.11, 137.58, 130.96, 129.19, 115.97, 113.77, 83.58, 74.03, 66.40, 55.24, 37.52, 35.65, 16.05, 10.75; HRMS m/e calcd. for (M⁺) C₁₇H₂₆O₃: 278.1881, found: 301.1766 (M+Na)

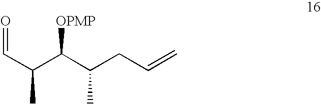

Preparation of 16: To a solution of the above compound 28 (1.0g, 3.59 mmol) in 150 ml CH₂Cl₂ was added pyridine (0.63 ml, 7.8 mmol) at 0° C. Dess-Martin periodinane (2.8 g, 6.5 mmol) was then added. The ice bath was then removed and the mixture was stirred for 3 h at room temperature. The reaction was quenched with 100 ml Na₂S₂O₃/NaHCO₃ (1:1) and extracted with CH₂Cl₂ (3×200 ml). The organic layer was washed with brine, dried (Na₂SO₄) and concentrated in vacuo. Purification of the residue by flash chromatography (silica, 4:1 hexane:EtOAc) afforded 16 (953 mg. 96%). [α]_{D}=−22.3° (c=0.52, CHCl₃); IR (film): 3162.5, 2925.2, 2366.1, 1719.2, 1513.6, 1396.0, 1243.3, 1129.8, 1043.5 cm⁻¹; ¹H NMR (500 MHz, CDCl₃) δ 9.78 (s, 1H), 7.19 (d, J=8.8 Hz, 2H), 6.84 (d, J=8.8 Hz, 2H), 5.75 (m, 1H), 5.03 (m, 2H), 4.36 (m, 2H), 3.78 (s, 3H), 3.68 ( dd, J=3.0, 8.1 Hz, 1H), 2.56 (dq, J=2.6, 14.0 Hz. 1H), 2.41 (m, 1H), 1.94 (dt, J=8.1, 16.9 Hz, 1H), 1.84 (m, 1H), 1.16 (d, J=7.0 Hz, 3H), 0.89 (d, J=7.0 Hz, 3H); ¹³C NMR (125 MHz; CDCl₃) δ 204.81, 159.22, 136.87, 130.19, 129.30, 116.51, 113.77, 81.49, 73.28, 55.26, 49.09, 37.23, 35.85, 15.94, 7.73; HRMS calcd. for (M⁺) C₁₇H₂₄O₃: 276.1725; found: 299.1622 (M+Na).

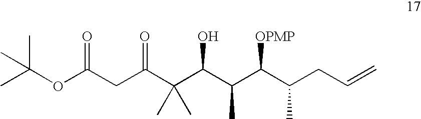

Preparation of 17: To a suspension of NaH (24 mg 60% dispersion, 0.6 mmol) in 2.5 ml anhydrous THF was dropwise added the t-butyl β-keto ester (99.4 mg, 0.53 mmol) in 1.2 ml THF at 0° C. The mixture was stirred for 10 min at that temperature and n-butyllithium (0.35 ml, 1.6M, 0.56 mmol) was then added. The yellow solution was stirred at 0° C. for additional 10 min. A solution of 16 (159 mg, 0.58 mmol) in 2 ml THF (washed with additional 0.5 ml) was then added dropwise. The resulting mixture was slowly warmed to room temperature with stirring. The reaction was quenched with saturated NH₄Cl (10 ml) after 20 min and extracted with CH₂Cl₂ (3×30 ml). The combined organic layer was washed with brine, dried (Na₂SO₄), filtered and concentrated in vacuo. The residue was-purified by flash chromatography (silica, 16:1 to 4:1 hexane:EtOAc) to give the condensation product 17 (186 mg, 70%, 8:1 dr.) as a clear oil. [α]_{D}=3.9° (c=0.83, CHCl₃); IR (film): 2966.3, 2931.0, 1736.8, 1701.6, 1613.4, 1507.7, 1396.0, 1313.8, 1240.1, 1143.4, 1037.6 cm⁻¹; ¹H NMR (500 MHz, CDCl₃) δ 7.27 (d, J=8.8 Hz, 2H), 6.88 (d. J=8.8 Hz, 2H), 5.78 (m, 1H), 5.04 (m, 2H), 4.63 (d of AB, J=10.6 Hz, 1H), 4.46 (d of AB, J=10.6 Hz, 1H), 3.80 (s, 3H), 3.71 (d, J=2.2 Hz, 1H), 3.59 (d of AB, J=11.2 Hz, 1H), 3.49 (d of AB. J=11.2 Hz, 1H), 3.27 (dd, J=3.0, 7.4 Hz, 1H). 2.87 (d, J=2.2 Hz, 1H), 2.48-2.41 (m, 1H), 2.05-1.88 (m, 3H), 1.46 (s, 9H), 1.19 (s, 3H), 1.13 (s, 3H), 0.89 (d, J=6.6 Hz, 3H) 0.87 (d, J=7.0 Hz, 3H); ¹³C NMR (125 MHz, CDCl₃) δ 209.14, 167.30, 159.37, 137.19, 130.17, 129.50, 116.34, 113.96, 89.77, 81.37, 80.91, 74.15, 55.27, 52.38, 47.86, 37.35, 35.62, 35.08, 27.96, 22.55, 20.92, 15.94, 7.96; HRMS m/e calcd. for (M⁺) C₂₇H₄₂O₆: 462.2981; found: 485.2889 (M+Na)

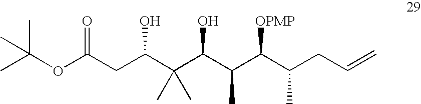

Preparation of 29: To a solution of tetramethylammonium triacetoxyborohydride (1.28 g, 4.87 mmol) in 3 ml CH₃CN₃ was added 3 ml AcOH, the mixture was stirred at room temperature for 30 min, cooled to −30° C., and treated with a solution of 17 (280 mg, 0.61 mmol) in 3 ml CH₃CN (washed with 1 ml). The reaction was stirred at −30° C. for 28 h and quenched with 30 ml saturated NaHCO₃ solution. The mixture was extracted with CH₂Cl₂ (3×100 ml). The organic layer was washed with brine, dried (Na₂SO₄) and concentrated in vacuo. Purification of the residue by flash chromatography (silica, 6:1 hexane:EtOAc) afforded the diol 29 (233 mg. 83%, 10:1dr). [α]_{D}=−2.9° (c=0.51, CHCl₃); IR (film): 3448.1, 3432.6, 2966.2, 2928.0, 1725.1, 1610.5; 1513.6, 1396.0, 1369.6, 1246.2, 1146.3, 1037.6 cm⁻¹; ¹H NMR (600 MHz, CDCl₃) δ 7.27 (d, J=8.3 Hz, 2H), 6.89 (d, J=8.7 Hz, 2H), 5.79 (m, 1H), 5.04 (m, 2H), 4.61 (d of AB, J=10.5 Hz, 1H), 4.49 (d of AB, J=10.5 Hz, 1H), 3.97 (m, 1H), 3.93 (d, J=4.8 Hz, 1H), 3.81 (s, 3H), 3.59 (d. J=2.2 Hz, 1H), 3.19 (dd, J=3.1, 7.5Hz, 1H), 3.16 (d, J=2.2 Hz, 1H), 2.45 (m, 1H), 2.41-2.34 (m. 2H), 2.04 (m. 1H), 1.97-1.87 (m, 2H), 1.47 (s, 9H), 1.02 (d, J=7.0 Hz, 3 H). 0.93 (s. 3H), 0.92 (d. J=7.0 Hz, 3H), 0.91 (s, 3H); ¹³C NMR (150 MHz, CDCl₃) δ 173.65, 160.29, 138.16, 131.11, 130.31, 116.92, 114.64, 90.67, 82.12, 81.36, 75.92, 74.64, 55.61, 41.03, 38.59, 37.53, 35.84, 35.18, 28.27, 21.88, 21.64, 16.29, 8.88; HRMS m/e calcd. for (M⁺) C₂₇H₄₄O₆: 464.3138; found: 487.3029 (M+Na).

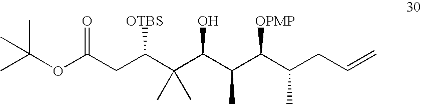

Preparation of 30: To a solution of 29 (310 mg, 0.668 mmol) in 70 ml anhydrous CH₂Cl₂ was added 2,6-lutidine (170 ul, 1.5 mmol) the mixture was cooled to −78° C. and TBSOTf (190 ul, 0.83 mmol) was then added dropwise. After 30 min, saturated NaHCO₃ (30 ml) was added. The mixture was extracted with CH₂Cl₂(3×100 ml). The organic layer was washed with brine, dried (Na₂SO₄) and concentrated in vacuo. The residue was purified by flash chromatography (silica, 5:1 hexane:EtOAc) to give the TBS silyl ether 30 (386 mg, 100%) as a clear oil. [α]_{D}=−13.1 (c=0.58, CHCl₃); IR (film): 3471.6, 3154.3, 2957.4, 2931.0, 2854.6, 3258.4, 2337.5, 1727.7, 1511.6, 1462.7, 1397.6, 1248.8, 1122.5, 1066.6 cm⁻¹; ¹H NMR (600 MHz, CDCl₃) δ 7.30 (d, J=8.3 Hz, 2H), 6.86 (d, J=8.8 Hz, 2H), 5.80 (m, 1H), 5.01 (m, 2H), 4.60 ( d of AB, J=10.6 Hz, 1H), 4.51 (d of AB, J=10.6 Hz, 1H), 4.10 (t, J=4.8 Hz, 1H), 3.80 (s, 3H), 3.75 (s, 1H), 3.58 (s, 1H), 3.20 (t, J=5.2 Hz, 1H), 2.66 (dd, J=4.8, 17.1 Hz, 1H), 2.38 (m, 1H), 2.34 (dd, J=5.3, 17.1 Hz, 1H), 1.97-1.89 (m, 3H), 1.45 (s, 9H), 1.03 (s, 3H), 1.02 (d, J=7.0 Hz, 3H), 0.98 (d, J=6.6 Hz, 3H), 0.90 (s, 9H), 0.78 (s, 3H), 0.15 (s, 3H), 0.09 (s, 3H); ¹³C NMR (150 MHz, CDCl₃) δ 171.49, 159.01, 137.98, 131.15, 12928, 115.71, 113.72, 113.70, 88.52, 80.72, 75.76, 74.08, 55.26 42.26, 40.25, 36.42, 36.19, 35.79, 28.10, 26.00, 21.84, 20.98, 18.08, 17.10, 10.00, −4.41, −5.02; HRMS m/e calcd. for (M⁺) C₃₃H₅₈O₆Si: 578.4002; found: 601.3905 (M+Na).

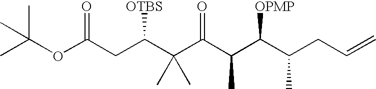

Fragment A

Preparation of fragment A: To a solution of the above compound 30 (386 mg, 0.67 mmol) in 40 ml CH₂Cl₂ was added pyridine (1.3 ml, 16 mmol) at 0° C. Dess-Martin periodinane (0.56 g, 1.3 mmol) was then added. The ice bath was then removed and the mixture was stirred for 3 h at room temperature. The reaction was quenched with 100ml Na₂S₂O₃/NaHCO₃ (1:1) and extracted with CH₂Cl₂(3×60 ml). The organic layers were combined and was washed with brine, dried (Na₂SO₄) and concentrated in vacuo. Purification of the residue by flash chromatography (silica, 4:1 hexane:EtOAc) afforded the title compound (348 mg. 90%). [α]_{D}=−31.0° (c=0.31, CHCl₃); IR (film): 2959.9, 2933.0, 2854.5, 1729.5, 1694.3, 1512.1, 1465.1, 1366.7, 1243.3, 1155.1, 1084.6, 990.6, 831.9, 773.2 cm⁻¹;¹H NMR (500 MHz, CDCl₃) δ 7.03 (d, J=8.5 Hz, 2H), 6.87 (d, J=8.8 Hz, 2H), 5.72 (m, 1H), 4.99 (m, 2H), 4.50 ( d of AB, J=10.2, 1H), 4.42 (d of AB, J=10.3 Hz, 1H), 4.31 (dd, J=4.1, 5.2 Hz, 1H), 3.80 (s, 3H), 3.46 (dd, J=4.8, 5.9 Hz, 1H), 3.33 (m, 1H),2.48 (dd, J=4.0, 17.2 Hz, 1H),2.36 (br d, J=11.8 Hz, 1H), 1.96-1.88 (m, 1H), 1.45 (s, 9H), 1.28 (s, 3H), 1.14 (d, J=7.0 Hz, 3h), 1.09 (s, 3H), 0.96 (d, J=7.0 Hz, 3H), 0.88 (s, (H), 0.12 (s, 3H), 0.08 (s. 3H); ¹³C NMR (125 MHz, CDCl₃) δ 217.63, 171.21, 159.13, 137.51, 130.94, 129.41, 115.96, 113.73, 84.22, 80.53, 75.01, 74.25, 55.27, 53.49, 44.79, 41.26, 36.92, 35.85, 28.15, 26.02, 23.01, 20.53, 18.17, 17.60, 13.63, −4.38, −4.72; HRMS m/e calcd. for (M⁺) C₃₃H₅₆O₆Si: 576.3846; found: 599-3724 (M+Na).

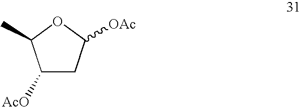

Preparation of bis-acetate 31: To a solution containing 13 (12.2 g, 103 mmol) and pyridine (27 ml, 0.33 mol) in 700 ml CH₂Cl₂ was added AcCl (22 ml, 031 mol) at 0° C. The ice bath was removed and the mixture was stirred for 30 min at room temperature. Water (300 ml) was added and the mixture was extracted with CH₂Cl₂ (3×500 ml). The combined organic layer was washed with brine, dried (Na₂SO₄) and concentrated in vacuo. Purification of the residue by flash chromatography (silica, 3:1 hexane:EtOAc) afforded bis-acetate (19.8 g, 95%). [α]_{D}=3.6° (c=1.61, CHCl₃) (α/β≈1.2); IR (film): 298.19, 1739.9, 1372.4, 1234.4, 1121.0, 1003.9; ¹H NMR (500 MHz, CDCl₃) δ α-anomer: 6.35 (dd, J=2.6, 5.9 Hz, 1H), 5.05 (ddd, J=3.3, 4.4, 7.0 Hz, 1H), 4.23 (dq, J=2.9, 6.6 Hz, 1H), 2.47 (ddd, J=2.6, 6.6, 14.3 Hz, 1H), 2:31 (ddd, J=4.4, 5.8, 14.6Hz, 1H), 2.07 (s, 31H), 2.06 (s, 3H), 1.34 (d, J=6.6 Hz, 3H); β-anomer: 6.30 (d, J=5.2, 1H), 4.84 (ddd, J=2.6, 3.7, 7.7 Hz, 1H), 4.31 (dq, J=3.7, 6.8 Hz, 1H), 2.55 (ddd, J=5.5, 7.7, 15.1 Hz, 1H), 2.11 (m, 1H), 2.09 (s, 3H), 2.08 (s, 3H), 1.30 (d, J=6.6 Hz, 3H); ¹³C NMR (125 MHz, CDCl₃) δ 170.74, 170.65, 170.37, 170.21, 98.22, 97.87, 81.56, 81.16, 77.94, 77.41, 38.05, 37.83, 21.28, 21.26, 20.99, 20.95, 20.08, 18.87.

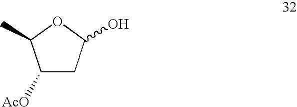

Preparation of 32: To a cooled (0° C.) solution of bisacetate 31 (2.19 g, 11 mmol) in CH₃CN (250 ml, 1 ml H₂O) was added BF₃.Et₂O (2.1 ml, 17 mmol). After 2.5 h, the reaction was quenched with saturated sodium bicarbonate solution (300 ml). After most organic solvent was removed, the residue was extracted with EtOAc (3×300 ml). The combined organic layer was washed with brine, dried (Na₂SO₄), filtered and concentrated in vacuo. Purification of the residue by flash chromatography (silica, 4:1 hexane: EtOAc) afforded the hemiacetal 32 (1.45 g, 84%). [α]_{D}=22.80 (c=4.0, CHCl₃) (α/β˜1.7); IR (film): 3428.9, 2977.3, 1734.0, 1441.8, 1372.8, 1248.0, 1069.1, 975.5; ¹H NMR (500 MHz, CDCl₃) δ α-anomer: 5.55 (d, J=4.8 Hz, 1H), 4.85 (ddd, J=2.6, 3.3, 7.4 Hz, 1H), 4.33 (dq, J=3.3, 6.2 Hz, 1H), 2.43 (ddd, J=5.5, 7.4, 14.7 Hz, 1H), 2.09 (s, 3H), 1.99 (ddd, J=1.1, 2.2, 14.7 Hz, 1H), 1.26 (d, J=6.3 Hz, 3H); β-anomer 5.62 (dd, J=4.1, 5.5 Hz, 1H), 5.04 (dt, J=3.3, 6.6 Hz, 1H), 4.12 (dq, J=3.0, 7.0 Hz, 1H), 2.30 (ddd, J=4.0, 6.6, 14.3 Hz, 1H), 2.19 (ddd, J=3.7, 5.5, 14.3 Hz, 1H), 2.06 (s, 3H), 1.37 (d, J=7.1 Hz, 3H); ¹³C NMR (125 MHz, CDCl₃) δ 170.77, 170.69, 98.57, 97.90, 80.22, 79.22, 78.85, 78.29, 39.24, 38.94, 21.03, 20.97, 20.62, 18.96; HRMS m/e calcd. for (M⁺) C₇H₁₂O₄: 160.0736; found: 183.0633 (M+Na)

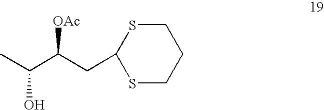

Preparation of dithane 19: lactol 32 (138 mg, 0.86 mmol) was then-dissolved in 10 ml CH₂Cl₂ and 1,3-propanedithiol (200 μl, 2.0 mmol) was then added to the solution. The resulting mixture was cooled to −78° C. TiCl₄ (123 μl, 1.12 mmol) was added. 30 min later, the reaction was quenched with saturated sodium bicarbonate solution (5 ml). The mixture was extracted with CH₂Cl₂ (3×50 ml). The combined organic layer was washed with brine, dried (Na₂SO₄), filtered and concentrated in vacuo. Purification of the residue by flash chromatography (silica, 4:1 hexane: EtOAc) afforded dithane alcohol 19 (222 mg, 97%). [α]_{D}=−11.1° (c=1.05, CHCl₃); IR (film): 3424.6, 2919.2, 1730.4, 1413.7, 1239.7, 1114.0, 1025.9; ¹H NMR (500 MHz, CDCl₃) δ 5.11 (dt, J=3.3, 8.6 Hz, 1H), 4.06 (dd, J=5.5, 9.5 Hz, 1H), 3.94 (m, 1H), 2.90-2.82 (m, 4H), 2.14 (s, 3H), 2.10 (m, 1H), 2.09 (dd, J=5.4, 8.6 Hz, 1H), 2.03 (ddd, J=2.9, 5.8, 15.0 Hz, 1H), 1.87 (m, 1H), 1.18 (d, J=6.5 Hz, 3H); ¹³C NMR (125 MHz, CDCl₃) δ 171.10, 75.00, 69.14, 43.68, 34.93, 30.18, 29.91, 25.66, 21.27, 17.90. ESI calcd for (M⁺) C₁₀H₁₈O₃S₂: 250; found: 251 (M+H), 273 (M+Na), 285 (M+Cl).

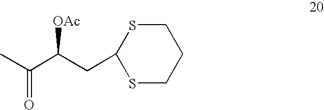

Preparation of ketone 20: To a cooled (−78° C.) solution of (COCl)₂ (1.5 ml, 17 mmol) in 150 ml CH₂Cl₂ was added slowly DMSO (0.6 ml, 8.5 mmol). The mixture was stirred for 30 min. A solution of 19 (1.2 g, 4.8 mmol) in 10 ml CH₂Cl₂ (washed with additional 2×5 ml) was added to the above reaction solution. After 3 h, triethylamine (2.5 ml, 18 mmol) was added, and the mixture was slowly warmed to room temperature. Water (100 ml) was then added. The mixture was extracted with CH₂Cl₂ (3×200 ml). The combined organic layer was washed with brine, dried (Na₂SO₄) and concentrated in vacuo. Purification of the residue by flash chromatography (silica, 6:1-5:1 hexane:EtOAc) afforded 20 (1.07 g. 95%). [α]_{D}=12.1° (c=0.89, CHCl₃); IR (film): 1738.2, 1422.7, 1369.4, 1225.5, 1118.8, 1038.9 cm⁻¹; ¹H NMR (600 MHz, CDCl₃) δ 5.26 (dd, J=3.5, 9.2 Hz, 1H), 4.08 (dd, J=5.7, 9.2 Hz), 2.91-2.79 (m, 4H), 2.27 (ddd, J=3.5, 8.8, 14.5 Hz, 1H), 2.21 (s, 3H), 2.20 (m, 1H), 2.17 (s, 3H), 2.14-2.09 (m, 1H), 1.96-1.88 (m, 1H); ¹³C NMR (150 MHz, CDCl₃) δ 204.44, 170.25, 75.61, 42.43, 35.68, 29.48, 29.17, 26.21, 25.56, 20.71; HRMS m/e calcd. for (M⁺) C₁₀H₁₆O₃S₂: 248.0541; found:271.0438 (M+Na)

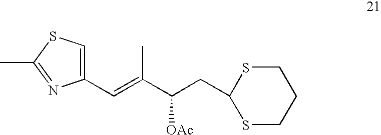

Preparation of 21: To a cooled (−78° C.) solution of phosphine oxide (62 1 mg, 2.0 mmol) in THF (15 ml) was added n-butyllithium (1.5 ml, 1.6M, 2.4 mmol). After 15 min, a solution of 20 (372 mg, 1.5 mmol) in 5 ml (washed with 2ml×2) THF was added slowly. The cooling dry ice bath was removed and the reaction mixture was allowed to warm to room temperature. Saturated NH₄Cl (20 ml) solution was added to quench the reaction. After most THF was evaporated. The mixture was extracted with EtOAc(3×50 ml). The combined organic layer was washed with brine, dried (Na₂SO₄) and concentrated in vacuo. Purification of the residue by flash chromatography (silica, 3:1 hexane:EtOAc) afforded 21 (452 mg, 88%). ¹H NMR (500 MHz, CDCl₃) δ 6.97 (s, 1H), 6.56 (s, 1H), 5.51 (dd, J=4.8, 7.6 Hz, 1H), 4.00 (t, J=7.4 Hz, 1H), 2.85 (m, 4H), 2.71 (s, 3H), 2.20 (m, 1H), 2.14-2.07 (m, 2H), 2.08 (s, 6H), 1.89 (m, 1H); ¹³C NMR (125 MHz, CDCl₃) δ 169.99, 164.70, 152.40, 136.63, 121.08, 116.64, 76.02, 43.19, 38.83, 29.81, 29.76, 25.76, 21.29, 19.24, 14.47 HRMS m/e calcd. for (M⁺) C₁₅H₂₁NO₂S₃: 343.0734; found:366.3706 (M+Na).

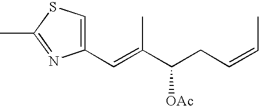

Fragment B

Preparation of fragment B: A solution of dithane 21 (268 mg, 0.78 mmol) was treated with CaCO₃ (105 mg, 1.05 mmol) and aqueous Hg(ClO₄)₂ (0.2M in H₂O, 4.8 ml, 0.96 mmol). The reaction mixture was stirred at room temperature for 2 h, treated with 30 ml ether, and stirred for 10 min. The precipitate was removed by filtration and the filtrate was diluted with H₂O (30 ml) and extracted with ether (3×50 ml) and dried over MgSO₄. The solvent was evaporated to afford a residue (220 mg).

A Solution of (Ph₃P⁺CH₂I)I⁻ (138 mg, 2.6 mmol) in THF (3 ml) at room temperature was added NaN(TMS)₂ (2.1 ml, 1M solution in THF, 2.1 mmol). At −78° C., the mixture was treated with HMPA (0.3 ml, 1.8 mmol) and the above crude aldehyde residue (220 mg in 3 ml THF). The reaction mixture was allowed to warm to room temperature and stirred for 1 h. After being quenched with saturated NH₄Cl (20 ml), the mixture was extracted with ether (3×50 ml). The combined organic layer was washed with brine, dried over Na₂SO₄ and concentrated in vacuo. Purification of the residue by flash chromatography (silica, 4:1 hexane:EtOAc) afforded fragment B (175 mg, 60%): [α]_{D}=−27.4° (c=1.36, CHCl₃); IR (film): 3154.3, 1731.0, 1396.0, 1225.6, 1190.4, 1114.0 cm⁻¹; ¹H NMR (500 MHz, CDCl₃) δ 6.97 (s, 1H), 6.54 (s, 1H), 6.35 (dt, J=1.5, 7.7 Hz, 1H), 6.18 (dd, J=6.9, 14.0 Hz, 1H), 5.40 (t, J 6.6 Hz, 1H), 2.71 (s, 3H), 2.66-2.53 (m, 2H), 2.10 (d, J=1.1 Hz, 3H), 2.09 (s, 3H); ¹³C NMR (125 MHz, CDCl₃) δ 170.06, 164.70, 152.33, 136.67, 136.28, 120.81, 116.48, 85.15, 38.44, 21.19, 19.21, 14.91; ESI calcd. for (M⁺) C₁₃H₁₆O₂NIS: 378; found: 378 (M⁺).

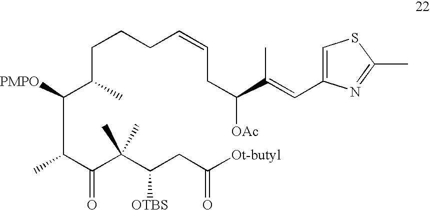

Preparation of 22: To a solution of fragment A (58 mg, 0.1 mmol) in 1.0 ml THF was added 9-BBN (0.5M in THF, 0.4 ml, 0.2 mmol). Water (0.1 ml) was added to the reaction mixture after 3 h. In a separated flask, fragment B (48 mg, 0.13 mmol) was dissolved in DMF (1.0 ml). Under vigorous stirring, CsCO₃ (60 mg, 0.18 mol), Ph₃As (5.6 mg, 0.018 mmol) and PdCl₂(dppf)₂ (15 mg, 0.018 mmol) were added sequentially. After stirring for 2 min, the quenched fragment A solution was added to the fragment B DMF solution quickly. After 8 h, the reaction mixture was poured into saturated NH₄Cl solution and extracted with CH₂Cl₂ (3×50 ml). The combined organic layer was washed with brine, dried (Na₂SO₄) and concentrated in vacuo. Purification of the residue by flash chromatography (silica, 4:1 hexane:EtOAc) afforded Suzuki coupling product 22 (54 mg. 65%). [α]_{D}=−32.9° (c=0.68, CHCl₃); IR (film): 2943.6, 2923.1, 1733.3, 1692.3, 1610.3, 1507.7, 1461.5, 1364.1, 1297.4, 1241.0, 1158.8, 1117.0, 830.8; ¹H NMR (600 MHz, CDCl₃) δ 7.29 (d, J=8.4 Hz, 2H), 6.94 (s, 1H), 6.86 (d, J=8.4 Hz, 2H), 6.51 (s, 1H), 5.47 (m, 1H), 5.31 (m, 1H), 5.27 (t, J=7.0 Hz, 1H), 4.48 (d of AB, J=10.3 Hz, 1H), 4.41 (d of AB, J=10.6 Hz, 1H), 4.30 (dd, J=4.4, 5.1 Hz, 1H), 3.79 (s, 3H), 3.41 (dd, J=4.4, 5.3 Hz, 1H), 3.31 (m, 1H), 2.70 (s, 3H), 2.51-2.41 (m, 3H), 2.17 (dd, J=5.7, 17.6 Hz, 1H), 2.06 (d, J=2.6 Hz. 3H), 2.05 (s, 3H), 2.01 (m, 1H), 1.55 (m, 1H), 1.45 (s, 12H), 1.27 (s, 3H), 1.22-1.16 (m, 2H), 1.12 (d, J=7.0 Hz, 3H), 1.07 (s, 3H), 0.94 (d, J=6.6 Hz. 3H), 0.87 (s, 9H), 0.12 (s, 3H). 0.07 (s, 3H). ¹³C NMR (150 MHz, CDCl₃) δ 217.75, 171.22, 170.19, 164.58, 159.07, 152.50, 137.27, 132.72, 130.96, 129.45, 123.98, 120.61, 116.20, 113.68, 84.62, 80.53, 78.50, 75.01, 74.17, 55.25, 53.43, 44.67, 41.23, 37.35, 31.01, 30.85, 28.13, 27.90, 27.49, 26.01, 22.98, 21.24, 20.44, 19.21, 18.15, 17.76, 14.80, 13.73, −4.39, −4.73; HRMS m/e calcd. for (M⁺) C₄₆H₇₃NO₈SSi: 827.4826; found: 850.4714 (M+Na),.

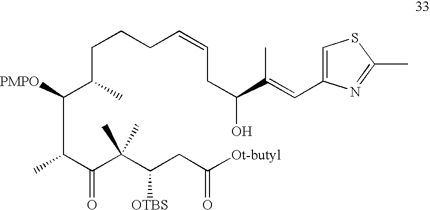

Preparation of 33: To a solution of 22 (10 mg, 0.012 mmol) in 1.5 ml MeOH was added catalytic amount MeONa at 0° C. The ice bath was removed and the solution was stirred at room for 2 h and quenched with 10 ml NH₄Cl. The mixture was extracted with CH₂Cl₂ (3×30 ml) The combined organic layer was washed with brine, dried (Na₂SO₄) and concentrated in vacuo. Purification of the residue by flash chromatography (silica, 4:1 hexane:EtOAc) afford alcohol 33 (8.1 mg, 85%), [α]_{D}=−26.5° (c=0.34, CHCl₃); IR (film): 3405.1, 2923.1, 1723.1, 1692.3, 1615.4, 1512.8, 1400.0, 1246.2, 153.8, 1112.8, 1066.7, 830.8; ¹H NMR (600 MHz, CDCl₃) δ 7.30 (d, J=8.4 Hz, 2H), 6.94 (s, 1H), 6.86 (d, J=8.4 Hz, 2H), 6.55 (s, 1H), 5.54 (m, 1H), 5.39 (m, 1H), 4.49 (d of AB, J=10.3 Hz, 1H), 4.40 (d of AB, J=10.6 Hz, 1H), 4.30 (t, J=4.5 Hz, 1H), 4.17 (t, J=6.5 Hz, 1H), 3.79 (s, 3H), 3.41 (t, J=5.1 Hz. 1H), 3.31 (m, 1H), 2.71 (s, 3H), 2.48 (dd, J=4.0, 17.6 Hz, 1H), 2.39 (m, 2H), 2.18 (dd. J=5.5, 17.2 Hz, 1H), 2.05 (m, 1H), 2.04 (s, 3H), 1.72 (d, J=3.0 Hz, 1H), 1.44 (s. 12H), 1.27 (s, 3H), 1.25 (m, 1H), 1.19 (m, 2H), 1.12 (d, J=6.6 Hz, 3H), 1.07 (s, 3H), 0.95 (d, J=6.6 Hz, 3H), 0.87 (s, 9H), 0.12 (s, 3 H), 0.07 (s, 3H); ¹³C NMR (150 MHz, CDCl₃) δ 217.71, 171.22, 164.50, 159.08, 152.84, 141.46, 133.21, 131.01, 129.45, 124.90, 119.05, 115.55,1 13.69, 84.61, 80.51, 74.95, 74.20, 55.25, 53.45, 44.66, 41.25, 37.35, 33.39, 30.91, 29.68, 28.14, 27.94, 27.56, 26.01, 22.99, 20.48, 191.8, 18.16, 17.73, 14.37, 13.74, −4.38, −4.72; HRMS m/e calcd. for (M⁺) C₄₄H₇₁NO₇SSi: 785.4720, found: 808.4636 (M+Na)

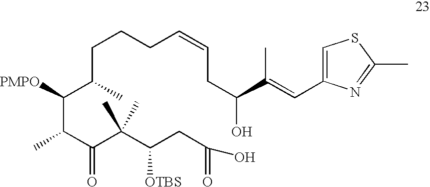

Preparation of 23: To a mixture of 33 (8 mg. 0.01 mmol) and 2,6-lutidine (35 μl, 0.3 mmol) in 1 ml CH₂Cl₂ at −78° C. was added dropwise TMSOTf (35 μl, 0.2 mmol). The dry ice bath was removed and the mixture was stirred at room temperature overnight. Saturated sodium bicarbonate solution (3 ml) was added. The mixture was extracted with CH₂Cl₂ (3×30 ml), and the combined organic layer was washed with brine, dried (Na₂SO₄) and concentrated in vacuo. The crude product was passed through a short silica pad (1:1 hexane:EtOAc) and the eluant was concentrated. The residue (6.6 mg, 0.019 mmol) in 2 ml MeOH was treated with 3 drops of 1N NaOH. After 3 h, 3 drops of 1N HCl were added to adjust the solution to neutral. The solvent was evaporated and the residue was purified by flash chromatography (silica, 4:1 hexane:EtOAc) to afford 23 (5.7 mg, 78%). [α]_{D}=−37.2° (c=0.25. CHCl₃); IR (film): 3365.8. 3180.8, 2931.0, 2860.5, 1703.5, 1613.5, 1512.5, 1460.S. 1396.0, 1249.3, 1090.0, 990.6, 833.3; ¹H NMR (500 MHz, CDCl₃) δ 7.31 (d, J=8.5 Hz 2H), 6.96 (s, 1H), 6.86 (d, J=8.5 Hz, 2H), 6.68 (s, 1H), 5.56 (m, 1H), 5.41 (m, 1H), 4.51 (d of AB, J=10.6 Hz, 1H), 4.44 (d, of AB, J=10.3 Hz, 1H), 4.43 (m, 1H), 4.18 (t, J=5.9 Hz, 1H), 3.79 (s, 3H), 3.46 (4.0, 5.7 Hz, 1H), 3.29 (m, 1H), 2.71 (s, 3H), 2.50 (br. d, J=15.4 Hz, 1H), 2.34 (m, 3H), 2.12 (m, 1H), 2.02 (m, 1H), 2.01 (s, 3H), 1.49 (m, 1H), 1.25 (s, 3H), 1.19 (s, 3H), 1.16 (s, 3H), 1.15 (d, J=6.6 Hz, 3H), 0.98 (d, J=6.6 Hz, 3H), 0.88 (s, 9H), 0.12 (s, 3H), 0.81 (s, 3H); ¹³C NMR (125 MHz, CDCl₃) δ 217.86, 171.24, 167.38; 159.04, 152.36, 141.86, 133.66, 131.03, 129.43, 124.83, 118.56, 113.68, 84.31, 74.73, 55.26, 54.15, 43.99, 37.19, 33.39, 30.94, 29.69, 27.86, 27.51, 26.01, 23.30, 18.90, 18.22, 17.50, 14.84, 14.60, −4.10, −4.66; HRMS m/e calcd. for (M⁺) C₄₀H₆₃NO₇SSi: 729.4094; found: 752.3971 (M+Na).

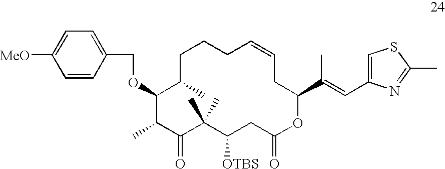

Preparation of 24: To a solution of 23 (5.7 mg, 0.0078 mmol) in THF (400μl) was added triethylamine (21 μl, 0.015 mmol) and 2,4,6-trichlorobenzoyl chloride (19 μl, 0.012 mmol). The mixture was stirred at room temperature for 20 min, diluted with toluene (0.6 ml), and added slowly over a period of 4.0 h to a solution of DMAP (64 mg, 0.53 mmol) in 10 ml toluene. After complete addition, the mixture was stirred for an additional 1 h and the solvent was evaporated in vacuo. The residue was purified by flash chromatography (silica, 6:1-3:1 hexane:EtOAc) to afford 24 (4.7 mg, 85%). [α]_{D}=−0.9° (c=0.24, CHCl₃); IR (film): 2828.7, 2855.1. 1737.6, 1696.2, 1604.7, 1512.2, 1461.6, 1383.4, 1250.0, 1162.7, 1107.5, 822.0; ¹H NMR (500 MHz, CDCl₃) δ 7.40 (d, J=8.4 Hz, 2H), 7.05 (s, 1H), 6.99 (d, J=8.4 Hz, 2H), 6.63 (s, 1H), 5.64 (dt, J=3.7, 11.4 Hz, 1H), 5.49 (m, 1H), 5.10 (d, J=10.6 Hz. 1H), 4.75 (d of AB, J=10.3 Hz, 1H), 4.64 (d of AB, J=10.6 Hz, 1H), 4.12 (d, J=10.2 Hz, 1H), 3.91 (s, 3H), 3.80 (d, J=9.5 Hz, 1H), 3.23 (m, 1H), 2.92-2.87 (m, 2H), 2.81 (s, 3H), 2.75 (dd, J=10.6, 16.5 Hz, 1H), 2.48 (m, 1H), 2.20 (s, 3H), 2.14 (dd, J=4.8, 12.8 Hz, 1H), 1.96 (m, 1H), 1.73 (m, 4H), 1.30 (m, 7H), 1.27 (s, 3H), 1.07 (d, J=6.6 Hz, 3H), 0.96 (s, 9H), 0.23 (s, 3H), 0.10 (s, 3H),; ¹³C NMR (150 MHz, CDCl₃) δ 215.55, 172.09, 165.54, 159.96, 153.36, 139.16, 135.93, 132.04, 130.16, 123.42, 120.74, 117.25, 114.60, 87.78, 80.80, 77.35, 76.69, 56.15, 54.24, 48.76, 39.54, 37.71, 32.51, 30.16, 29.18, 27.07, 25.89, 24.87, 21.15, 20.12, 19.51, 18.12, 15.54, 15.00, −2.18, −5.01; HRMS m/e calcd. for (M³⁰ ) C₄₀H₆₁NO₆SSi: 711.3989; found: 712.4051 (M+H).

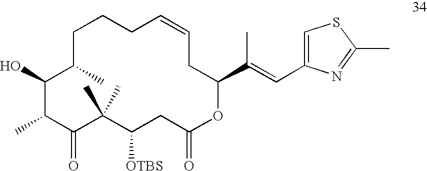

Preparation of 34: To a solution of 24 (4.7 mg, 0.0066 mmol) in dichloromethane (containing 5% H₂O, 2 ml) was added DDQ (4.0 mg, 0.018 mmol) at room temperature. After 3 h, the mixture was quenched with saturated NaHCO₃ solution. The mixture was extracted with CH₂Cl₂ (3×20 ml). The combined organic layer was washed with brine, dried (Na₂SO₄) and concentrated in vacuo. Purification of the residue by flash chromatography (silica, 5:1-3:2 hexane:EtOAc) afforded alcohol 34 (3.9 mg, 99%). [α]_{D}=−65.00 (c=0.48, CHCl₃); IR (film): 3424.6, 2919.2, 1860.5, 1736.9, 1689.8, 1460.7, 1378.4, 1149.3, 1096.4, 831.9; ¹H NMR (600 MHz, CDCl₃) δ 6.97 (s, 1H), 6.56 (s, 1H), 5.46 (dt, J=3.0, 10.9 Hz, 1H), 5.37 (m, 1H), 5.04 (d, J=10.3 Hz, 1H), 4.07 (t, J=6.2 Hz, 1H), 3.94 (t, J=2.9 Hz, 1H), 3.05 (m, 1H), 2.80 (br d, J=6.2 Hz, 2H), 2.71 (s, 3H), 2.35 (m, 1H), 2.11 (s, 3H), 1.99 (m, 1H), 1.78 (m, 1H), 1.25 (m, 7H), 1.17 (s, 6H), 1.14 (d, J=6.4 Hz, 3H), 1.01 (d, J=7.0 Hz, 3H), 0.83 (s, 9H), 0.12 (s, 3H), −0.04 (s, 3H); ¹³C NMR (125 MHz, CDCl₃) δ 217.98, 170.89, 164.65, 152.43, 138.24, 134.64, 124.08, 119.63, 116.07, 79.05, 76.31, 53.54, 43.04, 39.12, 38.81, 33.57, 31.96, 29.69, 28.43, 27.86, 26.15, 24.76, 22.93, 19.19, 18.61, 16.47, 15.27, 14.10, −3.59, −5.42; HRMS m/e calcd. for (M⁺) C₃₂H₅₃NO₅SSi: 591.3414; found: 592.3470.

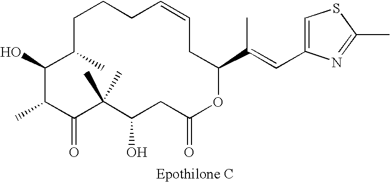

Preparation of epothilone C: To a solution of 34 (3.9 mg, 0.0066 mmol) in anhydrous THF in a plastic vial was added 0.5 ml HF.pyr complex at 0° C. The mixture was stirred overnight at room temperature and then diluted with 3 ml CHCl₃, which was added slowly to a precooled saturated NaHCO₃ solution (10 ml). The quenched mixture was extracted with CHCl₃ (3×30 ml). The combined organic layer was washed with brine, dried (Na₂SO₄) and concentrated in vacuo. Purification of the residue by flash chromatography (silica, 3:1-7:3 hexane:EtOAc) afforded alcohol epothilone C (3.1 mg, 95%). [α]_{D}=−81.2° (c=0.31, CHCl₃); IR (film): 3449.0, 2927.3, 2860.5, 1732.3, 1689.8, 1460.7, 1378.4, 1255.2, 1155.1, 1049.4, 728.2; ¹H NMR (600 MHz, CDCl₃) δ 6.96 (s, 1H), 6.59 (s, 1H), 5.44 (dt, J=4.4, 10.1 Hz, 1H), 5.38 (dt, J=4.9, 10.0 Hz, 1H), 5.28 (d, J=8.3 Hz, 1H), 3.72 (s, 1H), 3.40 (s, 1H), 3.04 (s, 1H), 2.70 (s, 3H), 2.72-2.64 (m, 1H), 2.48 (dd, J=11.4, 14.9 Hz, 1H), 2.33 (dd, J=2.2, 14.9Hz, 1H), 2.26 (br d, J=12.7Hz, 1H), 2.20-2.16 (m, 1H), 2.07 (s, 3H), 2.04-1.97 (m, 1H), 1.77-1.73 (m, 1H), 1.68-1.63 (m, 1H), 1.33 (s, 3H), 1.24 (m, 6H), 1.18 (d, J=7.0 Hz, 3H), 1.07 (s, 3H), 0.99 (d, J=7.0 Hz, 3H); ¹³C NMR (150 MHz, CDCl₃) δ 220.62, 170.37, 165.01, 151.96, 138.67, 133.43, 125.01, 119.35, 115.76, 78.37, 74.10, 72.29, 53.37, 41.64, 39.25, 38.57, 32.44, 31.77, 27.56, 27.44, 22.73, 19.05, 18.61, 15.94, 15.49, 13.45; HRMS m/e cacld. for (M⁺) C₂₆H₃₉NO₅S: 477.2549; found: 478.2631 (M+H).

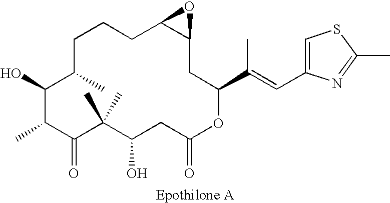

Preparation of epothilone A: To a solution of epothilone C (3.0 mg, 0.0064 mol) in 1 ml CH₂Cl₂ was added freshly prepared 3,3-dimethyldioxirane (0.5 ml in acetone, 0.045 mmol). The resulting solution was cooled to −30° C. for 3 h. A stream of argon was then bubbled through the solution to remove excess DMDO. The residue was purified by flash chromatography (silica, 6:4-1:1 hexane:EtOAc) to afford epothilone A (1.4 mg, 45%). [α]_{D}=−45.2° (c=0.14, MeOH); IR (film): 3389.3, 2919.2, 2848.7, 1731.0, 1689.8, 1454.8, 1384.3, 1260.9, 1119.9, 796.7; ¹H NMR (600 MHz, CDCl₃) δ 6.98 (s, 1H), 6.60 (s, 1H), 5.43 (dd, J=2.2, 8.4 Hz, 1H), 4.20 (m, 1H), 3.95 (br. d, J=6.2 Hz, 1H), 3.80 (dd of AB, J=4.1, 8.1 Hz, 1H), 3.23 (m, 1H), 3.04 (m, 1H), 2.90 (m, 1H), 2.70 (s, 3H), 2.58 (br, 1H), 2.54 (dd, J=10.6, 14.3 Hz, 1H), 2.41 (dd, J=3.3, 14.7 Hz, 1H), 2.13 (m, 1H), 2.09 (d, J=0.8 Hz. 3H), 1.88 (dt, J=8.5, 16.5 Hz, 1H), 1.79-1.71 (m, 2H), 1.37 (s, 3H), 1.28-1.20 (m, 5H), 1.18 (d, J=6.6 Hz, 3H), 1.11 (s, 3H), 1.00 (d, J=7.0 Hz, 3H); ¹³C NMR (150 MHz, CDCl₃) δ 220.35, 170.58, 165.13, 151.83, 139.02, 119.90, 116.21, 76.71, 74.5, 73.22, 57.49. 54.61, 52.89, 43.37, 38.93, 36.22, 31.46, 30.54, 27.17, 22.69, 21.54, 19.11, 17.09, 15.26, 14.10; HRMS m/e cacld. for (M⁺) C₂₆H₃₉NO₆S: 493.2498; found: 494.2561 (M+H).

Although the invention has been described with reference to the above examples, it will be understood that modifications and variations are encompassed within the spirit and scope of the invention. Accordingly, the invention is limited only by the following claims.

What is claimed is:

1. A method for producing an enantiomerically pure pyranose, comprising
A method for producing an enantiomerically pure pyranose, comprising
contacting a first achiral aldehyde, a second achiral aldehyde, and a third achiral aldehyde with 2-deoxyribose-5-phosphate aldolase (DERA) or a variant thereof under conditions suitable to facilitate sequential
asymmetric aldol reactions,
wherein a first aldol reaction between the first and second achiral aldehydes forms a first reaction product,
wherein a second aldol reaction between the first reaction product and the third achiral aldehyde forms a second reaction product,
wherein the second reaction product spontaneously undergoes an intramolecular cyclization reaction to form an enantiomerically pure pyranose.

2. The method of claim 1, further comprising oxidizing the enantiomerically pure pyranose under conditions suitable to produce an enantiomerically pure lactone.

3. The method of claim 1, wherein the first reaction product is a β-hydroxy-aldehyde.

4. The method of claim 3, wherein the β-hydroxy-aldehyde has the structure: 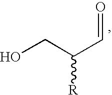
wherein R is —H, —OH, —N₃, —OMe, —CH₃, —CH₂CH₂N₃, CH₂CH₂OH, alkyl, aryl, or alkoxy.

5. The method of claim 1, wherein at least one of the first, second, or third achiral aldehydes is acetaldehyde.

6. The method of claim 1, wherein the enantiomerically pure pyranose has the following structure: 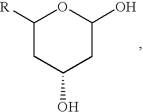
wherein R is —H, —OH, —N₃, —OMe, —CH₃, —CH₂CH₂N₃, CH₂CH₂OH, alkyl, aryl, or alkoxy.

7. The method of claim 1, wherein the enantiomerically pure pyranose has the following structure: 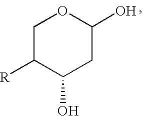
wherein R is —H, —OH, —N₃, —OMe, —CH₃, —CH₂CH₂N₃, CH₂CH₂OH, alkyl, aryl, or alkoxy.

8. The method of claim 1, wherein the second achiral aldehyde is an azide-containing acceptor aldehyde.

9. The method of claim 8, wherein an atorvastatin precursor molecule is produced.

10. The method of claim 8, wherein the acceptor aldehyde is 3-azidopropionaldehyde.

11. The method of any one of claims 1 to 10, wherein the 2-deoxyribose-5-phosphate aldolase variant has a mutation at amino acid residue 172, 205, 207, 238 or 239, or any combination thereof.

12. The method of claim 10, wherein the amino acid residue is 172 glutamic acid, 205 glutamic acid, 207 glutamic acid, 238 aspartic acid or 239 glutamic acid, or any combination thereof.
